(19) United States
(12) Patent Application Publication
(10) Pub. No.: US 2015/0284796 A1
(43) Pub. Date: Oct. 8, 2015

(54) Method to Identify Genes Relating to Improved Pathogen Resistance in Plants

(71) Applicant: The Penn State Research Foundation, University Park, PA (US)

(72) Inventors: James H. Marden, Lemont, PA (US); Claude W. dePamphiliis, State College, PA (US); Joshua P. Der, Fullerton, CA (US); Eric Kenneth Wafula, State College, PA (US); Mark Peterson, La Crescent, MN (US)

(21) Appl. No.: 14/677,035

(22) Filed: Apr. 2, 2015

Related U.S. Application Data
(60) Provisional application No. 61/974,666, filed on Apr. 3, 2014.

(51) Int. Cl. C12Q 1/68; G01N 33/00; G06F 19/22

(52) U.S. Cl. CPC C12Q 1/6876; G06F 19/22; G01N 33/0098; C12Q 2600/13; C12Q 2600/156

(57) ABSTRACT

The present invention provides a method for identifying one or more genes that harbor polymorphism or allelic variation likely to be important for resistance by plants to their pathogens. In certain embodiments, identification of the one or more genes provides guidance regarding how to modify an organism to exhibit a desired phenotype or for direct use as anti-biological compounds.

### CROSS-REFERENCE TO RELATED APPLICATIONS

This application claims priority to U.S. Provisional Patent Application No. 61/974,666 filed Apr. 3, 2014, the contents of which are incorporated by reference herein in their entirety.

### STATEMENT REGARDING FEDERALLY SPONSORED RESEARCH OR DEVELOPMENT

This invention was made with government support under DEB-1120476 awarded by the National Science Foundation. The government has certain rights in the invention.

### BACKGROUND OF THE INVENTION

Crop plants all over the world are threatened by an ever-evolving set of pathogens, which pose a major threat to security of the human food supply (Moffat, 2001, Science, 292: 2270-2273; Leach et al., 2001, Annu Rev Phytopathol, 39: 187-224; Gururani et al., 2012, Physiol Mol Plant Pathol 78: 51-65). Crop yields can be drastically reduced by pathogens, causing tremendous economic and social costs (McDonald and Linde, 2002, Ann Rev Phytopathol, 40: 349-379; McDowell and Woffenden, 2003, Trends Biotechnol, 21: 178-183), amounting sometimes to regional crop failures and starvation of human populations (e.g. the Irish potato famine). Plants resist pathogens by recognition of pathogen associated molecular patterns, PAMPs, which trigger general plant defense responses referred to as PAMP-triggered immunity (PTI). The plant genes encoding these recognition proteins are often called PRR genes. Pathogens are sometimes able to suppress the different components of PTI by ‘effector’ proteins delivered into the plant. A second line of plant defense involves the recognition of specific effectors (avirulence [Avr] proteins] by resistance (R-) genes within the plant, triggering effector-triggered immunity (ETI) (Boyd, 2012, Trends in Genetics, 29(4): 233-240).

Using these two (not entirely distinct) types of disease resistance genes (PR and R) plants detect pathogens and activate basal immunity and/or the hypersensitive response that limits the spread of infection, and ultimately kills pathogens (Boyd, 2012, Trends in Genetics, 29(4): 233-240; Dangl, 2013, Science, 341(6147): 746-751). Existing knowledge of R genes has already proven highly useful in breeding programs in use now for decades, and breeding for R gene mediated pathogen resistance is an efficient and environmentally friendly way to cope with plant disease (Boyd, 2012, Trends in Genetics, 29(4): 233-240; Dangl, 2013, Science, 341(6147): 746-751).

Efficient resistance breeding depends largely on understanding the genetic variation at R gene loci within the germplasm available for a given crop species and/or its wild relatives (McDowell and Woffenden, 2003, Trends Biotechnol, 21: 178-183; Boyd, 2012, Trends in Genetics, 29(4): 233-240; Dangl, 2013, Science, 341(6147): 746-751). For example, resistance to over 40 major diseases have been discovered in tomato wild relatives, and at least 20 of them have been bred into tomato cultivars (Ji et al. 2007, Mol Breeding, 20: 271-284; Robertson and Labate, 2007, Generic Improvement of Solanaceous Crops: Tomato, Volume 2, CRC Press, 25-75).

R genes are well known for often containing extremely high allelic diversity within and between populations, such as rpp5 (Noel et al. 1999, Plant Cell, 11: 2099-2111) and rpp13 (Bittner-Eddy et al., 2000, Plant J, 21: 177-188; Allen et al., 2004, Science, 306: 1957-1960; Rose et al., 2004, Genetics, 166: 1517-1527) in Arabidopsis, and L in flax (Ellis et al. 1999, Plant Cell, 11: 495-506). This allelic diversity and its relationship to pathogen resistance is best known in the model plant Arabidopsis thaliana (a fast-growing weed) and major food crops with annual generations and sequenced genomes. Rapid generational turnover permits large breeding experiments and QTL analyses, which in combination with a sequenced genome allows for high-resolution mapping to identify resistance alleles.

Many economically important plants do not yet have sequenced genomes, and in the case of woody plants (tree and woody vine crops), have generation times too long for rapid advances through breeding programs and QTL analyses.

Even for plants having a relatively short generation time, it is widely acknowledged that gene modification through breeding efforts is inefficient, as evidenced from the following quote from a recent publication regarding cassava, a plant having a short generation time: “The fact that cassava genome modification through breeding or engineering is time consuming and laborious necessitates a priori identification of the most promising resistance proteins” (Bart et al., 2012, Proc Natl Acad Sci USA. 109(28): E1972-E1979).

Thus, there is a need in the art for improved methods for identifying genes harboring allelic variation affecting or likely to affect for pathogen resistance. The present invention satisfies this unmet need.

### SUMMARY OF THE INVENTION

In one aspect the present invention provides a method of identifying one or more genes of an organism associated with pathogen resistance or pathogen response. The method comprises identifying the number of polymorphisms in each sequenced contig of a set of sequenced contigs, wherein the set of sequenced contigs are obtained from a pooled RNA sample from one or more sample organisms. The method comprises identifying each polymorphism located in the coding region of each sequenced contig as synonymous or non-synonymous polymorphism; and ranking the sequenced contigs based upon the presence of non-synonymous polymorphism in each sequenced contig. In one embodiment, the pooled RNA sample comprises the mRNA transcriptome from one or more sample organisms.

In one embodiment, the organism is a plant and where the polymorphisms are identified in each sequenced contig of a set of sequenced contigs obtained from a pooled RNA sample from one or more sample plants.

In one embodiment, the method comprises determining if each sequenced contig is a resistance (R) gene. In one embodiment, the method comprises determining if each sequenced contig is a pathogenesis-related (PR) gene. For example, in one embodiment, identifying putative R genes or PR genes among the sequenced contigs comprises utilizing homology and hidden markov models.

In one embodiment, the method comprises filtering the set of sequenced contigs to provide a set of orthology-established sequenced contigs. In one embodiment, the method comprises determining for each sequenced contig the most homologous gene of a model organism.

In one embodiment, the identified polymorphisms are single nucleotide variations (SNVs), multiple nucleotide variations (MNVs), and insertion-deletion polymorphisms. In one embodiment, the sequenced contigs are ranked by the ratio: (number of non-synonymous polymorphisms per non-synonymous site/number of synonymous polymorphisms per synonymous site) (pN/pS).

In one embodiment, the method comprises constructing a data structure comprising data for each of the sequenced contigs. In one embodiment, the data for each of the sequenced contigs comprise at least one of nucleotide sequence, contig name, contig length, read depth, normalized read depth, homologous gene of model organism, description of homologous gene, R gene status, PR gene status, predicted peptide sequence, location of polymorphism, type of polymorphism, number of polymorphisms, number of synonymous polymorphisms, number of non-synonymous polymorphisms, ratio of the number of non-synonymous polymorphisms to the number of synonymous polymorphisms, ratio of number of non-synonymous polymorphisms per non-synonymous site/number of synonymous polymorphisms per synonymous site, ratio of expression under an experimental condition to expression under control condition, ratio of expression under first experimental condition to expression under a second experimental condition, and number of non-synonymous mutations adjusted for contig length and read depth.

In one embodiment, the method comprises comparing the expression level of each sequenced contig in a first sample to the expression level of each sequenced contig in a second sample. In one embodiment, the first sample is of one or more sample organisms subjected to a treatment and the second sample is of one or more sample organisms not subjected to a treatment.

In one embodiment, the method comprises aligning sequenced contigs from two or more species to locate trans-specific amino acid polymorphisms associated with pathogen resistance.

In one embodiment, the identified one or more genes are used to modify an organism to provide the organism with enhanced pathogen defense. In one embodiment the organism is modified by at least one of gene editing, introduction of a transgene, or direction of a breeding program. In one embodiment, modification of the organism also modifies the descendants of the organism. In one embodiment, the identified one or more genes is used in the development of an anti-biological compound.

In one aspect the present invention provides a system for identifying one or more genes of an organism associated with pathogen resistance or pathogen response, the system comprising a computing device running a software platform. The software program is configured to identify the number of polymorphisms in each sequenced contig of a set of sequenced contigs, wherein the set of sequenced contigs are obtained from a pooled RNA sample from one or more sample organisms; identify each polymorphism located in the coding region of each sequenced contig as synonymous or non-synonymous polymorphism; and rank the sequenced contigs based upon the presence of non-synonymous polymorphism in each sequenced contig.

In one embodiment, the software platform is configured to construct a data structure comprising data for each of the sequenced contigs, wherein the data for each of the sequenced contigs comprise at least one of nucleotide sequence, contig name, contig length, read depth, normalized read depth, homologous gene of model organism, description of homologous gene, R gene status, PR gene status, predicted peptide sequence, location of polymorphism, type of polymorphism, number of polymorphisms, number of synonymous polymorphisms, number of non-synonymous polymorphisms, ratio of the number of non-synonymous polymorphisms to the number of synonymous polymorphisms, ratio of number of non-synonymous polymorphisms per non-synonymous site/number of synonymous polymorphisms per synonymous site, ratio of expression under an experimental condition to expression under control condition, ratio of expression under first experimental condition to expression under a second experimental condition, and number of non-synonymous mutations adjusted for contig length and read depth.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is a schematic demonstrating that in certain aspects, the present invention is directed to identification of polymorphic R genes displaying a high level of non-synonymous mutations. Identification of such genes originates from obtaining genetic information of most or all of expressed genes in a sample, and in certain instances includes filtering such information to establish a large set of orthology-established genes.

FIG. 2 is a flow chart depicting an exemplary method of the present invention.

FIG. 3 depicts an exemplary data structure comprising data from sequenced contigs produced by the method of the invention. Depicted is a ranked list of candidate genes harboring signals of diversifying selection. The first column is the name of the Genus/species gene model (partially obscured here); the next three columns show the relative expression level of that gene under three different experimental treatments; column 5 shows the yes/no status (0/1) of the outcome of blast and HMM searches for R gene similarity; column 6 shows the orthogroup identity from an analysis of 22 plant genomes; column 7 shows the Pfam (protein family) of the orthogroup; columns 8 & 9 show the number of synonymous and non-synonymous polymorphisms in that gene model; columns 10-11 show the ratio of synonymous polymorphisms to synonymous sites (pS) and the ratio of non-synonymous polymorphisms to non-synonymous sites (pN). Column 12 shows the ratio of pN/pS, including a small Bayesian adjustment that prevents zero denominators from going to infinity. Additional columns not shown here include information such as the BLASTp homologous protein in a crop plant (Theobroma cacao for example) and the strength of that homology. This information could be used to select and clone alleles from these species loci for functional testing and eventual transgenic applications, or could be used to guide a search for alleles at the homologous locus in, for example, T. cacao or to make novel alleles by editing at sites homologous to non-synonymous sites in these sequenced and analyzed species. This list of highly ranked genes comes from an original list of ˜150,000 gene models from transcriptomes of six species, which included 2,219 R gene homologs.

FIG. 4 is a table showing a set of genes from six tree species that contain both an unexpectedly high level of non-synonymous polymorphism and are strongly upregulated by experimental treatment with the plant hormone salicylic acid (SA). This gene list exemplifies a method of the present invention, which combines expression and polymorphism data to identify functionally important candidate loci in pathogen response genes.

FIG. 5 depicts the results of analyses demonstrating the identification of trans-specific polymorphisms in various species. Dots represent some number of conserved or divergent amino acids; letters indicate amino acids present in two wild tree species (Eugenia mesiotica and Dipteryx oleifera) at sites where trans-specific polymorphism was identified. The top amino acid refers to the amino acid observed in the consensus sequence, where the bottom represents the minor allele. Also depicted are orthologous genes from two crop species (Theobroma cacao and Malus domestica), which, in certain cases, also contain one of the amino acids observed in the wild species at the analogous location.

FIG. 6 is a graph depicting the comparison of observed vs. predicted amplicon size for 23 highly polymorphic R genes from Virola (solid circles) and Beilschmiedia (open circles). The best fit line (green) corresponds closely with the line of identity (red).

FIG. 7 is a set of plots comparing metrics of R gene polymorphism from de novo transcriptome assemblies of tropical trees versus R gene polymorphism obtained from full genome sequences of two rice strains. Left panel: Non-synonymous polymorphism (pN; non-synonymous polymorphisms per non-synonymous site) in relation to synonymous polymorphism (pS; synonymous polymorphisms per synonymous site) in R genes of six species of tropical trees. Right panel: the equivalent measures (aka Ka, Ks) in two strains of rice (reproduced from Yang et al., 2006). Tree data are the subset of R genes in which pS>0.05; rice data show R genes with Ks>0.05 (i.e. these are the most polymorphic R genes in both trees and rice). The line of identity is shown in black. Genes in the remainder of the transcriptome (data not shown) tend to fall farther beneath the line of identity, presumably because of purifying selection.

FIG. 8 is a plot providing estimates of pN/pS within matrilines (vertical axis) in relation to the population-level estimates (horizontal axis) used in the analyses.

FIG. 9 is a plot showing the equivalence of pN/pS of R genes compared to species least-square mean pN.

FIG. 10 are a set of plots showing minor allele frequencies observed in Lacmellea panamensis. Data is separated into whole transcriptome-synonymous (upper left), whole transcriptome-nonsynonymous (upper right), R genes-synonymous (lower left), and R genes-nonsynonymous (lower right). Vertical axis shows N SPNs at each minor allele frequency in the transcriptome assemblies. By definition, the maximum possible frequency of the minor allele=0.50.

FIG. 11 are a set of plots showing minor allele frequencies observed in Dipteryx oleifera. Data is separated into whole transcriptome-synonymous (upper left), whole transcriptome-nonsynonymous (upper right), R genes-synonymous (lower left), and R genes-nonsynonymous (lower right). Vertical axis shows N SPNs at each minor allele frequency in the transcriptome assemblies. By definition, the maximum possible frequency of the minor allele=0.50.

FIG. 12 are a set of plots showing minor allele frequencies observed in Virola surinamensis. Data is separated into whole transcriptome-synonymous (upper left), whole transcriptome-nonsynonymous (upper right), R genes-synonymous (lower left), and R genes-nonsynonymous (lower right). Vertical axis shows N SPNs at each minor allele frequency in the transcriptome assemblies. By definition, the maximum possible frequency of the minor allele=0.50.

FIG. 13 are a set of plots showing minor allele frequencies observed in Beischmiedia pendula. Data is separated into whole transcriptome-synonymous (upper left), whole transcriptome-nonsynonymous (upper right), R genes-synonymous (lower left), and R genes-nonsynonymous (lower right). Vertical axis shows N SPNs at each minor allele frequency in the transcriptome assemblies. By definition, the maximum possible frequency of the minor allele=0.50.

FIG. 14 are a set of plots showing minor allele frequencies observed in Brosimum alicastrum. Data is separated into whole transcriptome-synonymous (upper left), whole transcriptome-nonsynonymous (upper right), R genes-synonymous (lower left), and R genes-nonsynonymous (lower right). Vertical axis shows N SPNs at each minor allele frequency in the transcriptome assemblies. By definition, the maximum possible frequency of the minor allele=0.50.

FIG. 15 are a set of plots showing minor allele frequencies observed in Eugenia nesiotica. Data is separated into whole transcriptome-synonymous (upper left), whole transcriptome-nonsynonymous (upper right), R genes-synonymous (lower left), and R genes-nonsynonymous (lower right). Vertical axis shows N SPNs at each minor allele frequency in the transcriptome assemblies. By definition, the maximum possible frequency of the minor allele=0.50.

FIG. 16 is a set of graphs showing mean (+/−SE) minor allele frequency for each species, categorized by R gene status and synonymous vs. nonsynonymous polymorphism. Only sites where the read depth was between 50 and 1000 are included. To keep the sample size constant, the number of R genes that met these coverage criteria in each species was first determined. Then, that number of genes from the remainder of each respective transcriptome was randomly subsampled, repeating 10,000 times to obtain each species' mean and standard error. Also shown is the entire transcriptome exclusive of R genes (i.e. no sub-sampling).

### DETAILED DESCRIPTION

The present invention provides a system and method for identifying one or more genes that harbor polymorphism or allelic variation likely to be important for resistance by plants to their pathogens. In certain embodiments, identification of the one or more genes provides guidance regarding how to modify an organism to exhibit a desired phenotype or for direct use as anti-biological compounds.

For example, in certain instances, the present invention provides a rapid and reliable method to obtain a list of one or more candidate genes and amino acids within such genes, for modification in a cultivar. In one embodiment, the list is generated based on the relative number of non-synonymous and synonymous polymorphisms observed in the nucleic acids obtained from a wild species or a pathogen resistant species.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention provides a method for identifying one or more genes that harbor a polymorphism or allelic variation that are likely to be associated with a particular phenotype of an organism. In certain embodiments, identification of the one or more genes provides guidance regarding how to modify an organism to exhibit a desired phenotype. For example, in certain embodiments, the method comprises obtaining, processing, and analyzing data for nearly all or all of the expressed genes in an organism in order to identify one or more of such genes that harbor a polymorphism or allelic variation that are likely to be associated with a particular phenotype of an organism.

In one embodiment, the present invention provides a method for identifying one or more genes in a plant, where the one or more genes harbor a polymorphism or allelic variation. In certain embodiments, the method produces a list of one or more genes of a plant likely to be associated with pathogen resistance, including for example R genes and pathogenesis-related (PR) genes. For example, the one or more identified genes may exhibit accumulated non-synonymous mutations associated with diversifying selection. For example, in certain embodiments, the method comprises obtaining, processing, and analyzing data for nearly all or all of the expressed genes of a plant in order to identify one or more of such genes that harbor a polymorphism or allelic variation that are likely to be associated with a pathogen resistance of the plant.

In one embodiment, identifying the one or more genes allows for using known technologies to edit the one or more genes in order to improve pathogen resistance in a plant of interest. For example, in one embodiment, the method uses a wild plant or a plant with resistance to one or more pathogens of interest in order to identify one or more genes associated with resistance. In certain embodiments, the method uses a wild plant or plant with resistance to one or more pathogens of interest to identify homologous gene loci likely to harbor important variation in a different species or cultivar (e.g. a crop plant). In one embodiment, the method is applied to the same species or cultivar in which improved pathogen resistance is desired in order to identify loci for marker-selected breeding. In one embodiment, the identified one or more genes are useful for guiding the genetic modification of a pathogen-susceptible species or cultivar in order to render the pathogen-susceptible plant resistant to the one or more pathogens of interest.

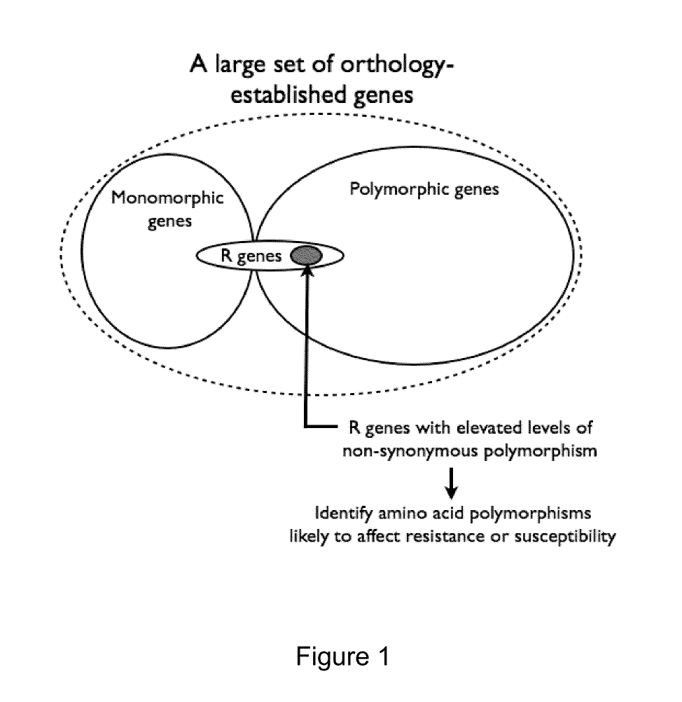

In certain embodiments, the method presented herein uses large pools of gene sequence data to ultimately extract information about gene expression and genetic variation in different treatment groups to discover the genes most likely useful for marker assisted breeding, gene editing, or transgenic manipulation to create durable resistance to pathogens. Candidate genes that are identified by way of the present invention can be used in transient transfection assays to determine if they convey resistance against specific pathogens or upregulate defense responses to specific pathogen effector molecules. For example, as depicted in FIG. 1, in certain embodiments, the method of the present invention uses various methods, described in detail elsewhere herein, to identify a subset of polymorphic R genes that display elevated levels of polymorphism.

It is important to note that identification of genes that are “likely” to harbor important variation represents an important advance because, although not providing certainty, it is far better than having no foreknowledge about which of the ˜25,000 protein coding genes in a plant are important for disease resistance, or which of the ˜200-600 R genes are most important. Identification of the top candidates is a significant improvement and a valuable starting point for further research.

In certain embodiments, the present method does not require a genome assembly and does not require a priori knowledge of the identity of the obtained nucleic acid molecules being analyzed. Further, in certain embodiments, the method uses population samples (i.e. diverse individuals potentially containing all or many segregating alleles) to provide models of all potential R genes and other genes that contain high levels of non-synonymous polymorphism and/or expression changes that indicate a likely role in pathogen resistance.

Crop plants have been selected from wild ancestral stocks and subjected to additional artificial selection and inbreeding. Some of this selection is for resistance to specific strains of particular plant pathogens, which greatly improves plant health and productivity in the short term, but any restriction of the breeding pool leaves a plant lineage more vulnerable to the diversity of pathogens that exist in nature and new strains of pathogens that may emerge.

The genetic mechanisms that determine plant resistance are well known. Plant immunity is determined in large party by a diverse group of resistance genes (R genes). R-genes generally consist of a nucleotide binding domain (NB) which binds by the conversion of ATP/ADP or GTP/GDP and a leucine rich repeat (LRR) domain generally involved in protein-protein interactions and ligand binding. Resistance is conveyed through various of mechanisms including, but not limited to:

a. The R protein interacts directly with an Avirulence gene product of a pathogen.

b. The R protein guards another protein that is degraded by an Avr gene.

c. The R protein may detect a pathogen/microbe-associated molecular pattern (PAMP or MAMP).

d. The R protein may direct enzymatic degradation of a toxin produced by a pathogen.

These interactions evolve rapidly, with pathogens selected to evade the actions of R genes, and R genes selected for effectiveness against new varieties of pathogens. This process, known in evolutionary biology as diversifying selection, leads to the accumulation of allelic variation at R gene loci in large outcrossed populations.

Other sets of genes are responders to signals initiated by R genes and other upstream components of signaling pathways activated in response to pathogens. Proteins encoded by these pathogenesis related (PR) genes interact directly with pathogens by degrading their cell walls (e.g. chitinases, glucanases) or inhibiting enzymes produced by the pathogens (e.g. polygalacturonase inhibiting proteins). These genes also can be subject to diversifying selection and have high levels of non-synonymous polymorphism.

In other words, the genes that interact directly with pathogens, either at the stage of detection or biochemical response, determine pathogen resistance and tend to exist as multiple forms within populations (i.e. alleles). This genetic diversity is severely reduced when a small number of plants are used to establish cultivars for agriculture, when particular traits are selected for propagation, and when there is inbreeding. Hence, practices used to select and propagate cultivars with favorable crop traits lead inevitably to reductions in pathogen resistance (i.e. reduction of allelic diversity at R and PR gene loci).

Using traditional selection methods, any grower can observe and select for disease-resistant plants or for any other trait for which there is heritable variation. However, in the absence of locally available variation in the trait of interest, this cannot be done. More sophisticated breeding methods use larger pools of material than available to a local grower. Ultimately, the most useful tool for managing plant traits, including pathogen resistance, is to identify the particular loci and alleles that underlie variation in the traits of interest, then genotype the potential breeding pool and make the appropriate crosses. The standard scientific approach for identifying genomic regions affecting pathogen resistance involves crossing a resistant to a susceptible strain and using genetic markers and offspring traits to perform genetic linkage mapping to identify chromosomal regions associated with resistance (called bulk segregant analysis or quantitative trait locus [QTL] mapping). These methods typically identify a genomic region containing dozens to hundreds of genes and so do not rapidly identify particular loci and alleles affecting the trait of interest. After considerable time (typically a number of plant generations) and expense to identify the critical genetic variants, the QTL approach makes it possible identify specific polymorphic sites responsible for pathogen resistance (e.g. Kumar 2014, Int Res J Biol Sci 3(1): 73-88). That information is used to genotype potential parent plants and select those with the alleles known to convey pathogen resistance (marker assisted breeding). Note that these methods are effective only for existing strains of problematic pathogens and have no ability to identify other genetic variation that may have been important in the past and possibly again in the future as pathogen strains move in and out of a given location and/or evolve.

Many crop plants (rubber, cacao, grape, citrus, apple, pear, and trees crops in general) have long generation times and therefore grow too slowly to be best suited for the multi-generational process presently used to identify alleles affecting pathogen resistance and other traits. Another limitation of current methods for selection of crop plant traits is that they depend on crosses of lineages within a single species or pairs of closely related species that can successfully interbreed (i.e. the inherent limitation of any method dependent on breeding).

As described herein, the present invention is based in part upon the development of a bioinformatic and functional genomic approach to identify and rank genes with potential resistance function from a single generation of plants, thus bypassing some of the time and expense of traditional breeding approaches. Further, because there is no breeding involved, the present invention allows for the utilization of the genetic diversity in wild plants or collections of different populations of cultivated plants, which dwarfs the reservoir of beneficial genetic variation in particular strains of crop plants. The present method improves upon previous methods which require pre-existing genomic data and/or neglect the absence of locally available variation in the trait of interest for identifying functionally important polymorphism that may be useful for improving crop plant traits, or use approaches that cannot characterize all R genes and are more expensive and time-consuming.

The method of the present invention is applicable to identifying potential functionally important genes (candidate genes) in any lineage, including wild species, which often grow alongside crops and share pathogens. Hence, the method opens the door to utilizing genetic variation from non-interbreeding strains and even wild species as either trans-genes or to guide the identification of important allelic variation at the homologous loci in various lineages of crop plants. This is an important breakthrough because other methods for improving plant pathogen resistance have made minimal use of the enormous pool of genetic variation in wild plants that cannot be interbred with crop plants. Hence, the method of the present invention may be useful and commercially valuable for improving crop plant health.

In certain embodiments, the method identifies one or more R or PR genes harboring a polymorphism associated with pathogen resistance. However, the present invention is not limited to identifying R or PR genes, as there are additional genes in plans that may be involved in pathogen defense which undergo diversifying selection. Therefore, the present invention may be used to identify any genes which may function in the response to pathogen or disease.

Users of this method will be able to rapidly survey cultivated plant strains or wild plants for genetic polymorphism likely to affect pathogen resistance and use the obtained material and information to 1) confirm enhanced resistance using transgenic approaches, including transient transgenic experiments in which individual leaves or other tissues are genetically manipulated and can confirm effectiveness rapidly; 2) marker assisted breeding to select for the identified and confirmed alleles; or 3) gene editing and/or transgenic manipulations for stable incorporation of functionally important alleles into a plant species or cultivar of interest (e.g. crop plants with known pathogen susceptibility). In certain embodiments, the present method identifies functionally important alleles in multiple loci so that marker assisted breeding or stable transgenic methods can create stacking (a.k.a. pyramiding) of resistance alleles. In certain instances, stacking is desirable because it prevents pathogens from rapidly evolving a solution to a single resistance mechanism (i.e. it enables more durable disease resistance).

In one embodiment, the candidate genes identified by the present method may be used as an anti-biological compound, including but not limited to an anti-bacterial, anti-viral, anti-fungal, anti-oomycete, anti-nematode, anti-insect, and the like. For example, in one embodiment, the method comprises identification of candidate genes that are involved in pathogen defense. It is demonstrated elsewhere herein, that the method can identify chitinase genes, which encode enzymes that break down the cell walls of fungi. Thus, the identified genes can be used for the development of a compound that may be used as a fungicide, or as a component of a fungicide. Such a fungicide can be used in a variety of agricultural, industrial, or commercial applications, including applications that do not necessarily involve plants. For example, the coding region of the identified genes can be used to produce one or more peptides or proteins having an anti-fungal property (i.e. chitinase activity). The present invention is not limited to identifying candidate genes having anti-fungal properties. Rather, the method allows identification of any polymorphic genes associated with pathogen defense that may be used to develop a compound useful to combat pathogens in any suitable application.

The present invention utilizes nucleic acid molecules collected from an individual plant or population of plants. In certain embodiments, the method utilizes nucleic acid molecules from different plants having known differences in phenotype (e.g. pathogen resistance). In one embodiment, the method utilizes nucleic acid molecules collected from one or more treated plants. Treated plants may be exposed to any suitable treatment, including for example, exposure to a pathogen, specific growing condition, temperature, humidity, soil, fertilizer, pesticide, and the like. In one embodiment, the treated plant is exposed to one or more compounds known or believed to increase expression of one or more defense response genes. Any type of nucleic acid molecule may be collected and used in the present invention.

The present method can be used to identify candidate genes that are likely involved in a trait of interest (e.g. pathogen resistance), in any suitable plant species. In certain instances, the method is used to identify candidate genes for slow-breeding plants for which traditional methods may be particularly unsuitable. Exemplary plants include, but are not limited to, varieties of woody food crop plants such as cacao, rubber, grape, citrus, apple, cherry, walnut, maple, oak, coffee, mango, papaya, pear, avocado, banana, walnut, pecan, pistachio, hazelnut, macadamia, apricot, peach, plum, grape, persimmon, mayhaw, and the like; varieties of woody fiber crops such as pine, poplar, cotton, and the like; and other crops such as corn, soy, wheat, rice, barley, hops, potato, melon, pineapple, tomato, tobacco, and the like. Such exemplary plants have too few generations per year for QTL and other breeding-based methods to provide rapid and relatively inexpensive results. Thus, the method presented herein may find application in any crop plant of interest.

In certain embodiments, the nucleic acid molecule is DNA, RNA, or a combination of DNA and RNA. In one embodiment, the nucleic acid molecule is mRNA. In certain embodiments, an entire transcriptome of the plant is collected. In certain embodiments, the method utilizes all or nearly all of the expressed genes (either in the form of DNA or RNA) to identify candidate genes, as described herein.

As contemplated herein, the present invention may be used in the analysis of any nucleic acid sample for which sequencing and analyses may be applied, as would be understood by those having ordinary skill in the art. For example, in certain embodiments, the nucleic acid can be, without limitation, genomic DNA, a subpopulation of DNA captured by annealing fragmented genomic DNA to well-designed probes matching coding regions only (exome sequencing), or targeted re-sequencing using PCR to amplify specific regions of genomic DNA. Other variations can include collection and analysis of mRNA. In one embodiment, reverse transcriptase is used to convert collected mRNA into DNA for subsequent analysis. In one embodiment, the mRNA is directly analyzed. In certain embodiments, DNA and/or RNA is obtained without a priori knowledge of its identity.

Nucleic acid molecules may be collected from some or all of the plant of interest. In certain embodiments, the nucleic acid molecules are isolated and collected from one or more tissues of interest. Examples of tissues of particular interest include but are not limited to roots, leaves, fruit and seeds.

In one embodiment, the method of the present invention uses the collected nucleic acid molecules, sequences it, assembles the transcriptome (i.e. constructs gene models of the coding regions of all genes expressed in the starting material), and uses bioinformatics, phylogenetics, gene polymorphism, and gene expression to organize the data, construct searchable databases, and obtain ranked lists of candidate genes containing potentially important polymorphism. In certain embodiments, traditional methods, such as PCR, cloning, long read sequencing of individual DNA molecules, and the like, can be used to characterize haplotypes at the candidate loci (i.e. fully characterize the potentially important gene variants).

In one embodiment, the method of the present invention comprises the building of databases and ranked lists of all protein coding gene regions containing variation most likely to affect the trait of interest (e.g. pathogen resistance). For example, in one embodiment, R genes and/or PR genes are identified that contain the most non-synonymous polymorphism relative to synonymous polymorphism (e.g. pN/pS or other measure of diversity in non-synonymous polymorphism or evolutionary rate of amino acid changes), an indicator of past and ongoing diversifying selection (i.e. evolutionary responses to diverse pathogens). In addition, if the starting material has been treated experimentally with pathogens or elicitors from pathogens, or comprises separate groups of susceptible versus resistant plants, ranked lists of genes most differently expressed or containing the non-synonymous allelic variants most strongly associated with resistance can readily be produced.

In certain embodiments, the method further comprises obtaining and analyzing microbial genes (e.g. bacterial, fungal, etc.) that may be associated with the plant. For example, the expression level and/or polymorphic analysis of microbial genes may be used to further refine a candidate list of plant genes, based on known or hypothesized interactions between the plant and microbial gene products.

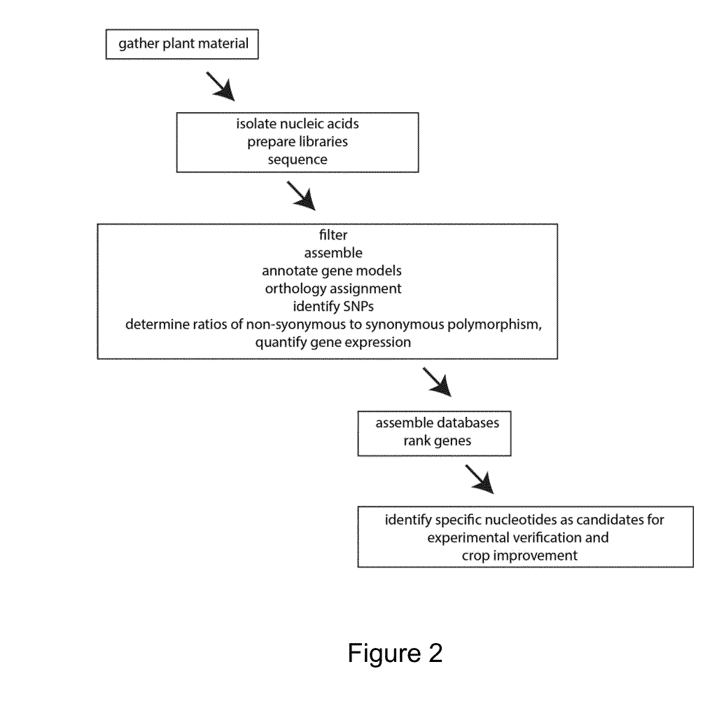

FIG. 2 depicts a flow chart describing an exemplary method of the present invention.

As described above, in one embodiment, the nucleic acid molecules are isolated and collected from one or more tissues of interest of one or more plants of interest. Methods for isolating and collecting nucleic acid from a tissue sample are well known in the art.

In one embodiment, the collected nucleic acid is prepared for large scale sequencing. In certain embodiments, all of the expressed genes are used for analysis. In another embodiment, a subset of the expressed genes is used for analysis. For example, PCR amplification and further sequencing of particularly interesting genes, for example genes showing high differential expression or pN/pS, may be used to verify or extend results of a larger scale sequencing effort. Sequencing of the nucleic acid molecules may be performed using any methodology or sequencing platform known in the art.

The nucleic acid may be prepared (e.g., library preparation) for massively parallel sequencing in any manner as would be understood by those having ordinary skill in the art. While there are many variations of library preparation, the purpose is to construct nucleic acid fragments of a suitable size for a sequencing instrument and to modify the ends of the sample nucleic acid to work with the chemistry of a selected sequencing process. Depending on application, nucleic acid fragments may be generated having a length of about 100-1000 bases. It should be appreciated that the present invention can accommodate any nucleic acid fragment size range, including whole molecules, that is appropriate for a sequencer. In one embodiment, this is done by dividing the reads into segments and assigning the read segments into abutting (but not overlapping) regions of analysis. This can be achieved by capping the ends of the fragments with nucleic acid adapters. These adapters have multiple roles: first to allow attachment of the specimen strands to a substrate (bead or slide) and second have nucleic acid sequence that can be used to initiate the sequencing reaction (priming). In many cases, these adapters also contain unique sequences (bar-coding) that allow for identification of individual samples in a multiplexed run. The key component of this attachment process is that only one nucleic acid fragment is attached to a bead or location on a slide. This single fragment can then be amplified, such as by a PCR reaction, to generate hundreds of identical copies of itself in a clustered region (bead or slide location). These clusters of identical DNA form the product that is sequenced by any one of several next generation sequencing technologies.

In certain embodiments, the nucleic acid molecules are analyzed by determining the sequence of individual molecules without the need of amplification, for example using a PacBio type method.

Next, the samples are sequenced using any standard massively parallel sequencing platform, as would be understood by those having ordinary skill in the art. For example, sequencers may include Roche 454, Illumina HiSeq 2000 or 2500, SOLiD, PacBio, Helicos, and the like. The present method also encompasses the use of sequencers and sequencing methodology developed in the future.

In one embodiment, the sequenced nucleic acid is assembled to create gene models. In certain embodiments, the sequenced nucleic acids are sequenced as multiple nucleic acid fragments. In certain instances, these sequenced fragments are referred to herein as “reads.” Any size of sequenced reads may be utilized to construct the gene model. For example, in certain embodiments, a model is assembled using overlapping regions of short reads (˜150 nucleotides). However, the present invention is not limited to the particular construction of the gene model. For example, in other embodiments, a gene model is assembled using sequenced reads of a large collection (thousands or more) of entire RNA molecules. Assembly of the gene model may comprise known techniques of processing sequenced reads. For example, in certain embodiments, the assembly of the gene model comprises pre-processing the sequenced reads to remove redundant and/or low quality sequences. In certain embodiments, the sequenced reads are trimmed to remove low quality bases and/or adapter sequences. In certain embodiments, the assembly of the gene model comprises normalization of the sequenced reads. In certain aspects, the assembled gene model is referred to as the “assembly” or “assembled sequences” comprising “predicted coding sequences” or “contigs.”

In certain embodiments, the method comprises assigning an orthology classification to the assembled sequences. For example, in certain instances the plurality of assembled sequences obtained from sequencing may contain redundant sequences, non-coding sequences, alternative-spliced variants, in the like which can confound subsequent analysis. Thus, in one embodiment, orthology analysis of the assembled sequences filters the gene models to reduce assembly artifacts. For example, in one embodiment, the gene models are compared against a set of annotated orthogroups, in order to assign each gene model into an orthogroup. In one embodiment, among very similar families of gene models, including gene models which are splice variants or those with intron retention, only one gene model is retained for analysis, thereby filtering out a great amount of redundancy. As demonstrated elsewhere herein, using orthology analysis to filter the assembled gene models drastically reduced the number of gene models by eliminating artifacts. However, the filtered models still demonstrate complete or near complete coverage of the transcriptome. Further, filtering of the assembled gene models, using orthology analysis, eliminates pseudogenes and introns that tend to have much higher rates of polymorphism than protein coding regions. Thus, in certain instances, if these pseudogenes and/or introns were left among the gene models for subsequent analysis, they can provide false signals of elevated levels of polymorphism and confound the ranked list of candidate genes.

In certain embodiments, the method comprises determining polymorphisms in the assembled sequences of the gene model. Polymorphisms may be detected using any platform or methodology known in the art. For example, various techniques may be implemented to detect any polymorphism, including, but not limited to single nucleotide variations (SNVs), multiple nucleotide variations (MNVs), insertion-deletion mutants, and the like. In certain embodiments, the parameters of polymorphism detection are optimized to provide more or less stringent variant detection. For example, in certain embodiments, non-specific reads and/or broken paired-end reads are ignored. In one embodiment, a polymorphism or variant is only considered if multiple reads are aligned to a site. The number of reads aligned to a site may be varied to alter the stringency of variant detection. Any method of variant detection may be used in the present method. Exemplary methods of variant detection include, but are not limited to, quality-based variant detection, probabilistic variant detection, structural variant detection, and the like.

In certain embodiments, the method comprises assigning each polymorphism as synonymous or non-synonymous. For example, in one embodiment, codon position is inferred and used to assign polymorphisms as synonymous or non-synonymous depending on whether the nucleotide change will affect the amino acid composition of the protein.

In one embodiment, the method comprises determining the homology of the gene models to genes of a functionally characterized species. For example, in one embodiment, the gene models are compared against the characterized genes of Arabidopsis thaliana. However, the present invention is not limited to the use of any particular species. Rather, the homology of the gene model assembled by way of the present invention may be determined using any characterized species, or species characterized in the future. Other exemplary functionally characterized species include, but are not limited to, various species of rice, tomato, and the like.

In certain embodiments, the method comprises identifying putative genes of a particular function. For example, in one embodiment, the method comprises using the assembled gene model to identify which genes or transcripts are putative R genes or PR genes. In one embodiment, the method comprises constructing or using a database of known R genes or PR genes, for example a BLAST protein database or a hidden Markov model (HMM) profile database, to classify the subsets of the assembled genes of the gene models as R genes and pathogen response genes. In certain embodiments, the method comprises assigning each gene model into an orthogroup, as described elsewhere herein. For example, in certain embodiments, gene models can be identified as R genes or PR genes if assigned to an orthogroup having a description including “disease,” “defense,” or “resistance.”

It should be understood by those skilled in the art that classification of assembled genes and/or determining the orthology and/or homology of assembled genes may be carried out by using the nucleotide sequence of the genes or the predicted amino acid sequence of the resultant peptide.

In certain embodiments, the expression differences for one or more genes across plants, species, treatment conditions, and the like are determined according to the number of sequences per gene, adjusted for gene length, within the assembled gene models. These differences can be related to experimental treatments or other differences (e.g. lineages) in the starting material. For example, the expression differences may be used to determine which genes respond transcriptionally to the treatments or have standing differences in lineages. Any method that counts the number observed sequences per gene accomplishes this task and thus may be used to evaluate the level of gene expression or to compare the levels of gene expression between samples. For example, in one embodiment, transcript abundance (FPKM or RPKM) is computed, with unique reads counted to matching transcripts. In certain embodiments, non-specifically mapped reads are either ignored or allocated on a proportional basis relative to the number of uniquely mapped reads.

In one embodiment, the method comprises construction of data structures that allow for sorting genes and/or searching genes using the bioinformatic information related to each assembled gene of the original sample or samples. For example, the data structures can comprise one or more of sequence data, expression level data, polymorphism data, orthology classification, homology, functional roles, and the like. In certain embodiments, the data structures rank the assembled genes by ordering those most likely to comprise one or more polymorphisms associated with a function or trait of interest. For example, in one embodiment, the data structures rank genes by their likelihood of harboring one or more polymorphisms that affect pathogen resistance. For example, if the starting material was obtained from a wild plant, the data structure may rank R genes and/or PR genes by the number or density of non-synonymous polymorphism deduced from the reading frame of the putative protein coding region.

In certain aspects, the method comprises obtaining information related to the level of polymorphism, including identifying the number and/or type of polymorphisms, the ratio of non-synonymous to synonymous polymorphisms, and the like, observed in the entire coding region of the gene models. In another embodiment, the method comprises obtaining information related to the level of polymorphism in pre-defined regions of the gene models. For example, in certain embodiments, the polymorphic analysis is restricted to leucine-rich regions.

These data structures allow searching by various criteria, including, but not limited to, the known functional roles of homologous genes, the orthology classification, and ranking according to quantitative variables such as expression level differences, the number of non-synonymous polymorphisms, or the ratio of non-synonymous to synonymous polymorphisms.

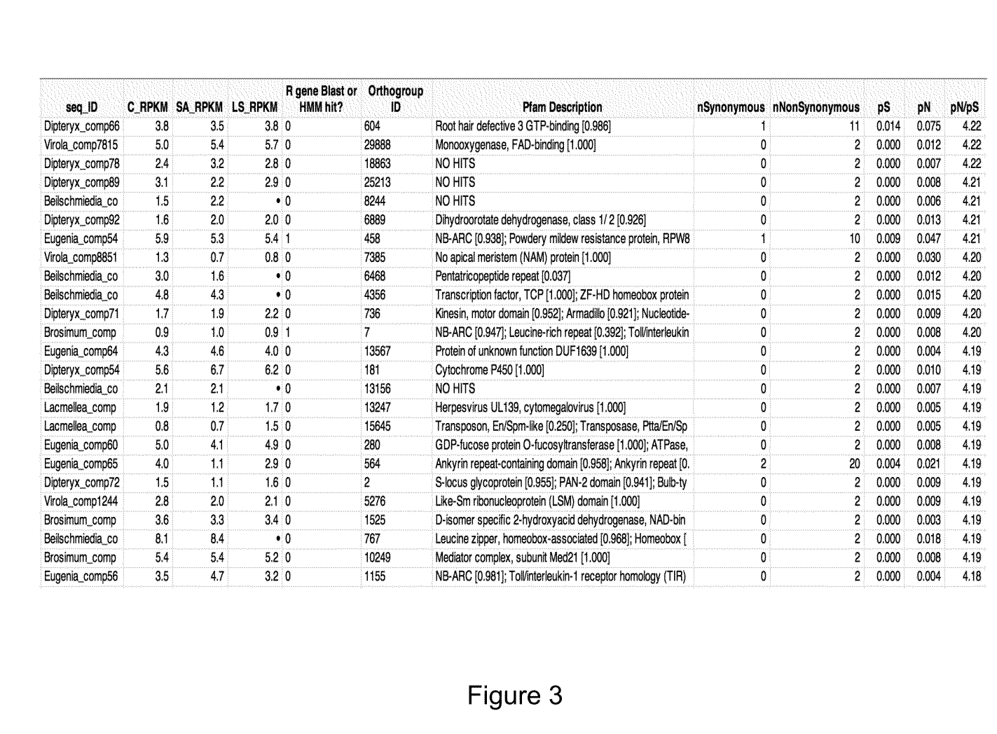

In one embodiment, the invention comprises constructing a data structure comprising data relating to one or more of the assembled gene models (contigs) of a particular sample or across all samples. An exemplary data structure is provided in FIG. 3. Exemplary data included in the constructed data structure, include but is not limited to, at least one of the following data for each assembled gene model (contig): genus/species, contig name, contig length, deduced coding sequence length, peptide length, average sequence depth, orthogroup identification, R-gene blast hit gene (yes/no), R-gene HMM hit (yes/no), PR gene (yes/no), the number of mapped reads, the number of normalized mapped reads, the number of single nucleotide variants in the coding sequence, the number of insertion/deletion variants in the coding sequence, log-transformed normalized sequence data, residuals of log transformed data that express gene-specific deviations in expression level, log transformed sequence length and depth.

In one embodiment, the data structure comprises data related to the expression level of the gene under various experimental conditions. For example, in one embodiment, the data structure comprises, for each gene model, a ratio of expression under an experimental condition to expression under control condition. In one embodiment, the data structure comprises, for each gene model, a ratio of expression under a first experimental condition to expression under a second experimental condition.

In one embodiment, the data structure comprises data of the number of synonymous and non-synonymous polymorphisms present in the contig, and/or the log-transformed number of the number of synonymous and non-synonymous polymorphisms present in the contig. In one embodiment, the data structure comprises pN, the ratio of non-synonymous polymorphisms to non-synonymous sites. In one embodiment, the data structure comprises pS, the ratio of synonymous polymorphisms to synonymous site. In one embodiment, the data structure comprises pN/pS, a composite ratio of pN to pS that represents the abundance of non-synonymous polymorphism relative to putatively neutral polymorphism present in the same gene. In one embodiment, the data structure comprises the ratio of the number of non-synonymous polymorphism to the number of synonymous polymorphism (dN/dS ratio). In one embodiment, the data structure comprises the Ka/Ks ratio, where Ka is the number of non-synonymous substitutions per non-synonymous site, and Ks is the number of synonymous substitutions per synonymous site. In one embodiment, the data structure comprises the ratio of non-synonymous to synonymous fixed differences in sequence alignments of the gene models against homologous sequences of another species or cultivar.

In certain embodiments, a Bayesian correction is applied to the dN/dS, pN/pS, or Ka/Ks ratio. This correction is equivalent to a low information prior expectation of neutral evolution. For example, in certain embodiments, 1 divided by the number of total sites was added to both pN and pS before calculating the pN/pS ratio. This is equivalent to the addition of one variant split at the same ratio as non-synonymous to synonymous sites (essentially, an estimate of the “next” mutation if polymorphisms are neutral). This small addition only had a substantive effect on genes with few identified variants, thereby reducing extreme values of little confidence and preventing infinity values when pS=0.

In certain instances, the ratio, pN/pS, dN/dS, or Ka/Ks, provides an indication on whether the gene model is a candidate gene that may affect pathogen resistance. For example, in certain instances, the gene model is a candidate gene when pN/pS is greater than about 15. In one embodiment, the gene model is a candidate gene when pN/pS is greater than about 10. In one embodiment, the gene model is a candidate gene when pN/pS is greater than about 8. In one embodiment, the gene model is a candidate gene when pN/pS is greater than about 5. In one embodiment, the gene model is a candidate gene when pN/pS is greater than about 4. In one embodiment, the gene model is a candidate gene when pN/pS is greater than about 3. In one embodiment, the gene model is a candidate gene when pN/pS is greater than about 2.5 In one embodiment, the gene model is a candidate gene when pN/pS is greater than about 2. In one embodiment, the gene model is a candidate gene when pN/pS is greater than about 1. In one embodiment, the gene model is a candidate gene when pN/pS is about 1. In certain embodiments, the ratio is calculated with respect to the entire coding region of the gene model. In certain embodiments, the ratio is calculated with respect to functional regions within the coding region, such as leucine rich regions in R genes.

In certain embodiments, the data structure includes data demonstrating the total number of unique single nucleotide variants and insertion/deletion polymorphisms across all contigs in a sample or across all samples. In one embodiment, such data is normalized or adjusted for other variables such as the number of total reads and/or the total assembly length. In certain embodiments, the data structure comprises output of blastx searches of the coding sequence against a model organism or other sequenced plant genome or transcriptome. In one embodiment, the data structure comprises functional annotation or description of the closest hit protein of the model organism or other sequenced plant genome or transcriptome.

In one embodiment, the present invention comprises constructing a data structure comprising data relating to one or more variants. In certain instances, the data structure comprises data relating to multiple variants of the same gene. In one embodiment, the variant data structure comprises data demonstrating at least one of, but not limited to, contig name, reference position, reference nucleotide, variant nucleotide, codon position, reference codon, variant codon, reference amino acid, variant amino acid, variant type (synonymous vs. non-synonymous), variant frequency, reference frequency, and quality.

Using the data structures described herein, genes can be searched and ranked for likely importance for pathogen resistance or for other trait of interest. For example, in certain embodiments, a ranked list of well supported genes containing the highest total number of non-synonymous sites (or highest number relative to the number of synonymous sites) is produced. In one embodiment, the data structure is ranked by pN/pS, dN/dS, or Ka/Ks. In one embodiment, the data structure is ranked by differential gene expression across treatments or between treatment and control. In certain embodiments, the ranking of gene models is done according a weighted sum of rankings based on pN/pS, differential expression, or other measure. Such a list reveals which putative resistance genes in the sample population contain potential functionally important variation, including variation that has been maintained by diversifying or fluctuating selection (a hallmark of pathogen resistance genes).

In one embodiment, the candidate genes are the top 5-1000 ranked contigs. In one embodiment, the candidate genes are the top 5-100 ranked contigs. In one embodiment, the candidate genes are the top 5-10 ranked contigs. In one embodiment, the candidate genes are the top 1-5 ranked contigs.

In another embodiment, contigs are first selected for expression level (e.g. those most upregulated by a treatment, pathogens, or pathogen-derived molecules) and that subset is ranked by pN/pS, dN/dS, Ka/Ks or other measures of the total or relative number of amino acid changing polymorphic sites. In another embodiment, the allele frequency of polymorphisms is taken into account so that common alleles are weighted more highly than rare alleles.

In another embodiment, gene models from two or more different species are analyzed. For example, in one embodiment, gene models from two or more species are analyzed for the presence of trans-specific polymorphisms in orthologous genes. Trans-specific polymorphisms may represent either convergent evolution of the same polymorphism or ancient alleles present in a common ancestor. In either case, these genes, and in particular the observed polymorphism in the genes, are particularly likely to affect pathogen resistance and can be classified as candidate genes.

In one embodiment, orthology analysis can be used to identify orthologous gene models across two or more plant lineages or species, and to further identify orthologous gene models that are ranked highly (i.e., high pN/pS, differential expression, etc.) in each of two or more lineages or species. The presence of highly ranked orthologous gene models across species provides further evidence that the associated gene is likely to affect pathogen resistance.

The data structures may be searched in a variety of ways. For example, if the treatment groups for RNA extraction included control and pathogen-exposed plants, it can be determined which genes with a particular orthology classification or functional annotation (for example, chitinase genes) were the most upregulated in response to pathogen exposure, and which among them contain the most non-synonymous polymorphism. The same can be done for the entire transcriptome, without reference to any particular functional annotation.

If the starting material comprises resistant and susceptible plants, the data structures can be searched for non-synonymous polymorphisms most strongly associated with resistance. There are many combinations of treatments of the original plant material, database searches and ranking criteria that can be performed using the methods described herein.

In one embodiment, ultimate use of the identified candidate genes may involve direct application of clones or edited versions of genes identified in the starting material (species or cultivar X) for use in species or cultivar X or in a different species or cultivar (species or cultivar Y). In one embodiment, the list of candidate genes may be used to direct further research on the homologous loci of species or cultivar Y. In the latter embodiment, gene loci showing signals of diversifying selection in species or cultivar X are used to infer the likely importance of alleles at the homologous loci in species or cultivar Y in order to avoid using transgenics (i.e. to guide the assessment of genetic variation in species or cultivar Y likely to have functional importance for pathogen resistance). For example, the method of the present invention could use wild citrus (or related wild species) to identify loci likely to be important for disease resistance. The identified genes could be cloned and used to transform commercial cultivars of citrus or to guide additional research to find or create (gene editing) variation in orthologous or homologous loci in commercial citrus cultivars. Here homologous refers to genes encoding proteins with about 35% or more identical amino acids over the length of the protein.

### System Platform

As contemplated herein, the present invention includes a system platform for performing the aforementioned methods for identifying one or more genes harboring polymorphisms likely to be associated with a desired trait, for example pathogen resistance.

In one embodiment, the system comprises instrumentation suitable for nucleic acid analysis, including, but not limited to, thermocyclers, sequencers, next generation sequencers, sequencer-associated equipment, library preparation kits, and the like.

In some embodiments, the method of the present invention may operate on a computer platform, such as a local or remote executable software platform, or as a hosted internet or network program or portal. In certain embodiments, only portions of the method may be computer operated, or in other embodiments, the entire method may be computer operated.

As contemplated herein, any computing device as would be understood by those skilled in the art may be used with the system, including desktop or moble devices, laptops, desktops, tablets, smartphones or other wireless digital/cellular phones, televisions or other thin client devices as would be understood by those skilled in the art. The platform is fully integratable for use with any sequencing platform and data output as described herein throughout.

For example, the computer operable component(s) of the system may reside entirely on a single computing device, or may reside on a central server and run on any number of end-user devices via a communications network. The computing devices may include at least one processor, standard input and output devices, as well as all hardware and software typically found on computing devices for storing data and running programs, and for sending and receiving data over a network, if needed. If a central server is used, it may be one server or, more preferably, a combination of scalable servers, providing functionality as a network mainframe server, a web server, a mail server and central database server, all maintained and managed by an administrator or operator of the system. The computing device(s) may also be connected directly or via a network to remote databases, such as for additional storage backup, and to allow for the communication of files, email, software, and any other data formats between two or more computing devices. There are no limitations to the number, type or connectivity of the databases utilized by the system of the present invention. The communications network can be a wide area network and may be any suitable networked system understood by those having ordinary skill in the art, such as, for example, an open, wide area network (e.g., the internet), an electronic network, an optical network, a wireless network, a physically secure network or virtual private network, and any combinations thereof. The communications network may also include any intermediate nodes, such as gateways, routers, bridges, internet service provider networks, public-switched telephone networks, proxy servers, firewalls, and the like, such that the communications network may be suitable for the transmission of information items and other data throughout the system.

Further, the communications network may also use standard architecture and protocols as understood by those skilled in the art, such as, for example, a packet switched network for transporting information and packets in accordance with a standard transmission control protocol/Internet protocol (“TCP/IP”). Any of the computing devices may be communicatively connected into the communications network through, for example, a traditional telephone service connection using a conventional modem, an integrated services digital network (“ISDN”), a cable connection including a data over cable system interface specification (“DOCSIS”) cable modem, a digital subscriber line (“DSL”), a T1 line, or any other mechanism as understood by those skilled in the art. Additionally, the system may utilize any conventional operating platform or combination of platforms (Windows, Mac OS, Unix, Linux, Android, etc.) and may utilize any conventional networking and communications software as would be understood by those skilled in the art.

To protect data, an encryption standard may be used to protect files from unauthorized interception over the network. Any encryption standard or authentication method as may be understood by those having ordinary skill in the art may be used at any point in the system of the present invention. For example, encryption may be accomplished by encrypting an output file by using a Secure Socket Layer (SSL) with dual key encryption. Additionally, the system may limit data manipulation, or information access. For example, a system administrator may allow for administration at one or more levels, such as at an individual reviewer, a review team manager, a quality control review manager, or a system manager. A system administrator may also implement access or use restrictions for users at any level. Such restrictions may include, for example, the assignment of user names and passwords that allow the use of the present invention, or the selection of one or more data types that the subservient user is allowed to view or manipulate.

As mentioned previously, the system may operate as application software, which may be managed by a local or remote computing device. The software may include a software framework or architecture that optimizes ease of use of at least one existing software platform, and that may also extend the capabilities of at least one existing software platform. The application architecture may approximate the actual way users organize and manage electronic files, and thus may organize use activities in a natural, coherent manner while delivering use activities through a simple, consistent, and intuitive interface within each application and across applications. The architecture may also be reusable, providing plug-in capability to any number of applications, without extensive re-programming, which may enable parties outside of the system to create components that plug into the architecture. Thus, software or portals in the architecture may be extensible and new software or portals may be created for the architecture by any party.

The system may provide software applications accessible to one or more users to perform one or more functions. Such applications may be available at the same location as the user, or at a location remote from the user. Each application may provide a graphical user interface (GUI) for ease of interaction by the user with information resident in the system. A GUI may be specific to a user, set of users, or type of user, or may be the same for all users or a selected subset of users. The system software may also provide a master GUI set that allows a user to select or interact with GUIs of one or more other applications, or that allows a user to simultaneously access a variety of information otherwise available through any portion of the system.

The system software may also be a portal or SaaS that provides, via the GUI, remote access to and from the system of the present invention. The software may include, for example, a network browser, as well as other standard applications. The software may also include the ability, either automatically based upon a user request in another application, or by a user request, to search, or otherwise retrieve particular data from one or more remote points, such as on the internet or from a limited or restricted database. The software may vary by user type, or may be available to only a certain user type, depending on the needs of the system. Users may have some portions, or all of the application software resident on a local computing device, or may simply have linking mechanisms, as understood by those skilled in the art, to link a computing device to the software running on a central server via the communications network, for example. As such, any device having, or having access to, the software may be capable of uploading, or downloading, any information item or data collection item, or informational files to be associated with such files.

Presentation of data through the software may be in any sort and number of selectable formats. For example, a multi-layer format may be used, wherein additional information is available by viewing successively lower layers of presented information. Such layers may be made available by the use of drop down menus, tabbed pseudo manila folder files, or other layering techniques understood by those skilled in the art or through a novel natural language interface as described hereinthroughout. Formats may also include AutoFill functionality, wherein data may be filled responsively to the entry of partial data in a particular field by the user. All formats may be in standard readable formats, such as XML. The software may further incorporate standard features typically found in applications, such as, for example, a front or “main” page to present a user with various selectable options for use or organization of information item collection fields.

The system software may also include standard reporting mechanisms, such as generating a printable results report, or an electronic results report that can be transmitted to any communicatively connected computing device, such as a generated email message or file attachment. Likewise, particular results of the aforementioned system can trigger an alert signal, such as the generation of an alert email, text or phone call, to alert a manager, expert, researcher, or other professional of the particular results. Further embodiments of such mechanisms are described elsewhere herein or may standard systems understood by those skilled in the art.

In one embodiment, the software platform of the system is configured for identifying one or more genes associated with pathogen resistance, using the method described elsewhere herein. For example, in certain embodiments, the software platform receives genomic or transcriptomic data from one or more samples to assemble gene models (contigs), assign models into orthogroups, identify gene homology, determine if the gene model is an R gene, determine if the gene model is a PR gene, identify the number and sites of synonymous polymorphisms, identify the number and sites of non-synonymous polymorphisms, calculate pN/pS, determine differential gene expression across treatments, compare gene models across species, or a combination thereof. In one embodiment, the software platform ranks the gene models according to gene expression, pN/pS, and/or other relevant measures discussed herein.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1

### A De Novo Transcriptomic Process to Accelerate the Identification of Genes for Pathogen Resistance and Crop Improvement

Presented herein is an example of analyses conducted by way of the method of the present invention. The described methods and results are from a study of polymorphism in disease resistance genes in trees growing wild in the Forest Dynamics Plot, Barro Colorado Island (BCI), Panama. It is demonstrated herein that the method produces a list of candidate genes with high likelihood to be R-genes and pathogen response genes harboring polymorphisms associated with pathogen resistance. Custom made scripts were created to perform the analyses presented herein. Exemplary commands are presented herein. However, the present invention is not limited to any particular script or command to carry out the method of the invention.

### Collection of Material, Seedling Rearing, and Experimental Treatments

The starting material for the process can be any living plant material from a sample of related plants in one or more populations of a species or variety of interest. The focal species used for these experiments are Beilschmiedia pendula, Brosimum alicastrum, Eugenia nesiotica, Lacmellea panamensis, Virola surinamensis, Dipteryx oleifera.

From beneath the canopy of five parent fruiting plants of each of these species, 15 post-dispersal seeds were collected. Cohorts of 15 seedlings per parent were grown in trays (one tray per parent plant per treatment) containing a common and well-mixed pool of sterilized soil obtained from within the forest. Three treatments were performed on this soil: a) control (sterile soil); b) soil inoculated 60 hrs. prior to harvest with a small quantity of live soil (an equal mix of subsamples collected from various locations in the forest); c) sterile soil soaked 48 hrs. Prior to harvest with the plant hormone salicylic acid (SA) (0.3 g of SA to 100 ml of distilled water per pot; volume of all pots were identical and weight approximately 1 kg. The purpose of these treatments was to stimulate changes in expression of genes that function in resistance to pathogens (present in live soil) and in response to the hormone (SA) that triggers the hypersensitive response. In the case of Beilschmiedia pendula, a high incidence of seed mortality caused by insect infestation reduced the abundance of live seeds to the point that two of the treatments (sterile soil; SA treated soil) were performed. After approximately one month of growth in a shade house, seedlings were uprooted, roots were washed free of soil, flash-frozen, then shipped for further analysis.

### Isolation of RNA and Preparation of Sequencing Libraries

Using the root sample from each individual seedling within a species, high quality RNA was isolated via a cetyltrimethyl ammonium bromide (CTAB) protocol (adapted from: Gambino et al., 2008., Phytochem Anal. 19:520-5). This yielded 75 RNA samples per species (except for Beilschmiedia, which had N=50), with mean RIN numbers of 7.5-8.4 and very little DNA contamination. This RNA was pooled within parent and treatment (5 seedlings per pool, 10-15 pools per species) using equal amounts of total RNA from each individual (800 ng quantified by RNA specific Qubit fluorometer). For each tree species, from this high-quality RNA (200 ng of total RNA per pool), bar coded libraries were prepared (TruSeq Stranded mRNA Sample Preparation Kit; N=15 per species), and combined in equimolar quantities for one lane per species of directional 2×150 bp paired-end sequencing on an Illumina HiSeq 2500 instrument. This yielded 212 to 262 million paired reads per species. After quality trimming and removal of duplicate reads, per-species read counts were 157 to 212 million (>22 GB of transcriptome data per species).

In general terms, this method provided expressed gene sequences from multiple offspring of multiple parents collected from outbred wild populations. The soil treatments affect gene expression, which are quantified at the level of seedling cohorts (maternal family; identifiable in sequences from the TruSeq bar code tags). Similarly, those tags can be used to identify maternal family-specific genetic polymorphisms.

### Sequence Cleaning, Assembly, and Annotation:

Transcriptome reads were trimmed to remove low quality bases and adapter sequences (Bolger et al., 2014, Bioinformatics, 30: 2114-2120), and then de novo assembled (one grand assembly of all reads within a species) using Trinity (Grabherr et al., 2011, Nature Biotechnology, 29: 644-652; Haas et al., 2013, Nature Protocols, 8: 1494-1512) with the e-normalization feature for large volume datasets. The pipeline includes methods found to improve the quality of de novo assemblies of large and complex transcriptome datasets, involving a BLAST-based process to initially cluster unigenes and then remove redundant or nearly identical or very lowly expressed sequences [e.g. (Wall et al., 2009, BMC Genomics, 10: 347)].

Pre-Processing of Illumina HiSeq Reads—Removal of Redundant and Low-Quality Sequences

Adapter and overrepresented sequences were identified in 10 randomly sampled 200,000 paired-end reads of each library using the FastQC (version 0.10.1) quality control program based on Illumina TruSeq version 3 (as used by HiSeq and MiSeq machines).

Adapter and overrepresented sequences were clipped from the reads, and low quality bases were trimmed using Trimmomatic (version 0.30) program.

In Silico Normalization of Pre-Processed Reads

The combined library reads for each species was normalized using the Trinity (release 20131110) in silico normalization program with default parameters except as indicated below (Haas et al., 2013, Nat Protoc. 8(8):1494-512).

De Novo Transcriptome Assembly of Normalized Reads

de novo transcriptome assembly was performed using the Trinity (release 20131110) program with all normalized reads for each species. Default parameters were used except as specified in the executed commands below (Grabherr et al., 2011, Nat Biotechnol, 29(7): 644-52).

### Deduced Coding Sequence and Peptide:

Post-Processing Assembled Contigs

Likely coding sequences in each species' de novo assemblies were identified using ESTScan (version 2.1). The positive strand coding sequences were filtered out (which comprised of less than 5% of the all predicted coding regions), and coding sequence cleanup and translation validation was performed (Iseli et al., 1999, ISMB, 99). Repeated coding sequences and sub-sequences were then filtered out using GenomeTools (version 1.5.1) to obtain non-redundant CDS (and peptide) sets for each of the species (Gremme et al., 2013, IEEE/ACM Transactions on Computational Biology and Bioinformatics, 1).

Sorting Plant Genes from the Non-Plant Genes

BLASTp was used to search the non-redundant predicted peptides of each species' post-processed assembly against the NCBI non-redundant protein database, followed by taxonomic classification of BLASTp hits using the Galaxy metagenomic analysis tool. This allowed for the identification of plant transcripts and for the relocation of the non-plant sequences to a different database for separate analyses (Altschul et al., 1990, J Mol Biol, 215.3: 403-410; Goecks et al., 2010, Genome Biol, 11.8: R86).

Orthology Analysis

The transcriptome assemblies initially contained about 150,000 plant gene models per species, with much redundancy caused by mRNA features such as alternative splicing. To overcome this redundancy and avoid artifacts such as intron retention, a well-validated orthology analysis (Wickett et al., 2014, Proc Natl Acad Sci USA, 111: E4859-4868; Honaas et al., 2013, BMC Plant Biology, 13: 9; Ming et al., 2013, Genome Biology, 14: R41; P. Amborella Genome, 2013, Science, 342, 1241089) was used that employs a Hidden Markov Matrix procedure to detect deep homology and assign gene models to orthogroups (narrowly defined gene families) from a global set of well-annotated orthogroups produced from 22 representative high quality plant genomes.

Clusters of narrowly-defined gene lineages representing evolutionarily related proteins (i.e. orthogroups) were constructed using the complete set of annotated proteins from 22 sequenced plant genomes selected to represent the breadth land plant diversity. The plant genomes analyzed in this classification include nine rosid genomes (Arabidopsis thaliana, Carica papaya, Fragaria vesca, Glycine max, Medicago truncatula, Populus trichocarpa, Thellungiella parvula, Theobroma cacao, Vitis vinifera), three asterids (Solanum lycopersicum, Solanum tuberosum, Mimulus guttatus), two basal eudicots (Nelumbo nucifera, Aquilegia coerulea), five monocots (Oryza sativa, Brachypodium distachyon, Sorghum bicolor, Phoenix dactylifera, Musa acuminata), one basal angiosperm (Amborella trichopoda), one lycophyte (Selaginella moellendorffii), and one moss (Physcomitrella patens).

Pairwise similarity among protein sequences was compared using all-by-all blastp searches (with an e-value cutoff of 1 e-5), followed by MCL-based clustering (Enlight et al., 2002, Nucleic Acids Research, 30: 1575-1584). Clustering may be implemented using several software packages, including but not limited to OrthoMCL (Li et al., 2003, Genome Res, 13: 2179-2189), ProteinOrtho (Lechner et al., 2011, BMC Bioinformatics, 12: 124), OrthoFinder (Steve Kelly Lab; Emms and Kelly, 2015, OrthoFinder: Solving fundamental biases in whole genome comparisons dramatically improves orthologous gene group inference accuracy; In Review). Clustering can vary in stringency to produce clusters that vary in size and inclusiveness, appropriate to various intended downstream applications in comparative genomics, including but not limited to phylogenetic analysis of gene families, identification of the evolutionary origin of new gene functions, reconstructions of gene and genome duplication events, and examination of the expansion and contraction of gene family size.

For the characterization of R-gene families, OrthoMCL (Li et al., 2003, Genome Res, 13: 2179-2189) was used with an inflation value of 3.5. Protein sequences in the resulting orthogroup clusters were aligned using the default parameters in MAFFT (Katoh et al., 2005, Nucleic Acids Research, 33: 511-518) and profile Hidden Markov Models (pHMMs) were constructed using the HMMER package (Eddy et al., 2009, Genome informatics International Conference on Genome Informatics, 23: 205-211; Finn et al., 2011, Nucleic Acids Research, 39: W29-37). Functional domains for each orthogroup were characterized and annotated using PFAM domain searches and retrieving associated GO terms.

Plant R-Gene Classification

Known R genes from the Plant Resistance Genes Database [PRGdb 2.0 (Sanseverino et al., 2013. Nucleic acids research 41.D1: D1167-D1171)] were used to build a BLAST database and 15 HMM profiles (Nawrocki and Eddy, 2013, Bioinformatics, 29: 2933-2935; Katoh and Standley, 2013, Molecular Biology and Evolution 30.4: 772-780) of R gene classes (Yu et al., 2014, BMC Genomics, 15: 3) that are defined by the presence of specific domains. Putative R genes were identified as those having a BLASTp or HMM match (1 e⁻⁵ cutoff) in the R Gene Database (Plant Resistance Gene Wiki) and a TAIR orthogroup description containing “disease”, “resistance”, or “defense”. Putative pathogenesis-related (PR) genes were obtained by BLASTp using sequences of known PR genes from Citrus (Campos et al., 2007, Genet Mol Biol, 30: 917-U198). Putative anti-oxidant genes in the fungal component of the non-plant genes were BLASTp identified using sequences from Chen et al., 2003, Molecular Biology of the Cell; 14: 214-229.

Homology Search and Functional Annotation Against Arabidopsis thaliana Protein Database

All known protein sequences from the Arabidopsis thaliana database were downloaded (The Arabidopsis Information Resource) and a BLAST protein database was constructed. Blastx was run using the plant portion of the transcriptome assemblies and retained the best hit for each query sequence. The A. thaliana best hit gene ID's were then used to associate plant contigs with Arabidopsis functional annotations (The Arabidopsis Information Resource).

### Expression Quantification (RNA-Seq):

Duplicate reads (coming from PCR amplification errors which can have a negative effect because a certain sequence is represented in artificially high numbers) were removed before reads were mapped onto gene models of the grand assembly using [CLC Genomics Workbench version 6.5.1]. For paired data, the assumption is made that if both ends of a paired read have sequences that are identical to both ends of another pair, then they are identified as duplicates, and only one copy is retained for subsequent analysis. The algorithm also takes sequencing errors into account during this filtering.

Transcript abundance, expressed as the number of reads (R) per kilobase of contig (K) per million sequence reads (M) (RPKM) was then computed from uniquely mapped reads, including the broken pair counting scheme.

Differential expression was calculated for each pair of treatments within a species using the Bioconductor package DESeq for R (Anders and Huber, 2010: Genome Biology 11:R106). Default parameters were used, including a 0.05 false discovery rate (FDR) cut off.

CLC Genomic Workbench (version 6.5.1) was used to perform RNA-Seq analysis and probabilistic variant detection, using the non-redundant predicted CDS of each species post-processed assemblies as the mapping reference.

### Polymorphism Detection:

Gene models assembled using all sequences within a species were analyzed using VarScan (Koboldt et al., 2009, Bioinformatics, 25: 2283-2285) to conservatively call single nucleotide (SNP) and insertion-deletion polymorphisms. A minimum read depth of 20 with at least 2 variant reads was required to support a variant call. Because samples were pools of many individuals and ample read depth was often observed, variants were called at frequencies as low as 0.5% to capture even rare alleles.

The variant calls for each cohort were combined into a single table with each gene and position represented on a single row. Number of variants per gene was calculated for each cohort and for total positions variable in each gene (including total variants and separately for SNVs and indels). Variability was further assessed for each gene by calculating the residual of log 10 N variants after correcting for gene length and average read depth.

To determine the synonymous versus non-synonymous status of each SNV, the codon containing each variable position was retrieved from the predicted coding sequence. The encoded amino acid was determined for the codon using both the reference (most common nucleotide at that position in the assembly) and the variant allele. SNV's that produced the same amino acid were called synonymous; those that produced different amino acids were called non-synonymous. This was performed in a custom R function utilizing tools from the package seqinr (Charif and Lobry, 2007, Structural Approaches to Sequence Evolution, 207-232). Both third-position bias and non-synonymous to synonymous ratios were then calculated for each gene as an estimate of selection on genes of various classes.

The reading frame was determined and used R (2014, R Foundation for Statistical Computing, Vienna, Austria) including the packages rnaseqWrapper (Peterson et al., 2015, CourseSource) and seqinr for R (Charif and Lobry, 2007, Statistical approaches to sequence evolution, 207-232; Springer) with the codon model of Li (Li et al., 1993, Journal of Molecular Evolution, 36: 96-99) to determine pN and pS for each gene model in comparison to a haplotype created in silico to represent all SNP variants in that gene. pN is the ratio of non-synonymous polymorphisms to non-synonymous sites; pS is the equivalent ratio for synonymous polymorphisms, and pN/pS is a composite ratio that represents the abundance of non-synonymous polymorphism relative to putatively neutral polymorphism present in the same gene. The distinction between synonymous and non-synonymous (for both sites and polymorphisms) can be blurred by the presence of additional polymorphism in the codon. To handle this complication, the number of synonymous and non-synonymous sites and polymorphisms were probabilistically distributed (for example, a position with simple ambiguity had a count of one-half added to each synonymous and non-synonymous). A Bayesian correction, equivalent to a low information prior expectation of neutral evolution (i.e., pN/pS=1), was applied to pN/pS for gene models containing at least one SNP site (invariant genes were ignored). To accomplish this, 1 divided by the number of total sites was added to both pN and pS before calculating the pN/pS ratio. This is equivalent to the addition of one variant split at the same ratio as non-synonymous to synonymous sites (essentially, an estimate of the “next” mutation if polymorphisms are neutral). This small addition only had a substantive effect on genes with few identified variants, thereby reducing extreme values of little confidence and preventing infinity values when pS=0.

Removal of Potentially Mis-Assembled Gene Models

The orthology analysis described above filters out gene models that have retained introns and/or transposons, yielding a highly filtered set of gene models. Even so, there were some clear outliers for polymorphism (pS>0.5; N=257, distributed roughly equally among the six species). Included in this set, in all six species, were genes with repeated motifs (e.g. polyubiquitin), but only two R genes. These hyperdiverse gene models were excluded from further analysis.

### Analyses of Gene Expression and Polymorphism

Methods described above provided input data for each species comprising:
Read counts for each gene for each cohort;
Calculated RPKM for each gene for each cohort (fragments per kilobase of transcript per million fragments mapped);
Identified single nucleotide variants (SNVs) and insertion-deletion (indel) variants for each cohort;
The predicted coding regions for each gene model; and
The BLAST output, including which genes aligned to other plant species.

A custom R script was used to merge the read and RPKM data from each cohort into a single table file.

Data Processing

All data analysis was conducted using custom R scripts calling several packages for the R statistical environment.

Basic Calculations

Average read depth for each gene in the assembly as a whole was then calculated based on the total reads for each gene divided by gene length. Using treatment groups within each set of cohorts (i.e. the three soil treatments for 15 seedlings of maternal cohort ‘A’ within species ‘B’), the mean RPKM (both raw and log 2; a measure of gene expression) was calculated. This was repeated for pooled data of treatment (i.e. ignoring cohorts and using data from seedlings of each treatment). From these data cohort (maternal family) and treatment effects on gene expression level is determined.

The results of the experiments and analyses are now described.

Identification of Pathogen Resistance Genes Containing High Levels of Non-Synonymous Variation and Differential Expression in Response to Live Soil and/or Salicylic Acid

To confirm that resistance gene homologs in the focal species contain elevated levels of non-synonymous polymorphism (log 10 N non-synonymous sites+0.1), a mixed model analysis was run that controls for log-transformed contig length and read depth) and categorizes all contigs as belonging to one of four groups:
not an R gene and the A. thaliana homolog lacks annotation for defense or disease resistance
not an R gene but the A. thaliana homolog has defense or disease resistance annotation
R gene homology but the A. thaliana homolog lacks annotation for defense or disease resistance
both R gene homology and the A. thaliana homolog has annotation for defense or disease resistance

The last of these categories (i.e. both R gene homology and homolog has notation for defense or disease resistance) is of particular interest.

For the six tree species analyzed to date (Beilschmiedia pendula, Brosimum alicastrum, Eugenia nesiotica, Lacmellea panamensis, Virola surinamensis, Dipteryx oleifera), there is significantly more non-synonymous variation in R genes than other genes across the remainder of the transcriptome (P<0.001 in all six cases).] This result confirms that non-synonymous polymorphism accumulates in the pathogen resistance genes of plants in general, which was not previously well documented outside of model species and a few crops.

### Discovery of Genes Likely to Affect Pathogen Resistance

In one analysis, genes were ranked according to dN/dS and most elevated in expression in a live soil inoculation treatment compared to a salicylic acid hormone treatment. The analysis was done to identify genes affected by exposure of roots to microbes but that are not SA induced genes. Upon ranking, the top ranked gene happened to be orthologous to a gene recently discovered to determine susceptibility of a woody plant to a fungus. In this case, for interesting physiological reasons, the fungus favors a high expression level of this gene and appears to seize control from the plant.

This gene that was top-ranked by the described unbiased approach, was subsequently shown by other researchers (independent of the present experiment and analyses) to affect pathogen resistance. Further analyses demonstrated that multiple species have polymorphism at a particular amino acid site or adjacent site in this gene. For example, in this particular gene, Brosimum and Dipteryx have a consensus sequence containing a SEVV motif, while Virola has a consensus sequence containing a GIVL at an analogous location. It was observed that polymorphisms existed for all of these species in the analyzed gene models. A Brosimum variant had a V to F mutation; a Dipteryx variant had a V to L mutation, and a Virola variant had a T to I mutation. This data can be used to infer that this motif is a likely site for useful marker assisted breeding or gene editing in a tree crop. For example, analysis of the gene models from this gene of a citrus crop indicates the presence of an AV sequence analogous to the EV of consensus Brosimum and Dipteryx and TV of consensus Virola. This AV sequence may thus be a candidate for gene editing to improve pathogen resistance in the citrus crop.

### Identification of Genes Related to Disease Response

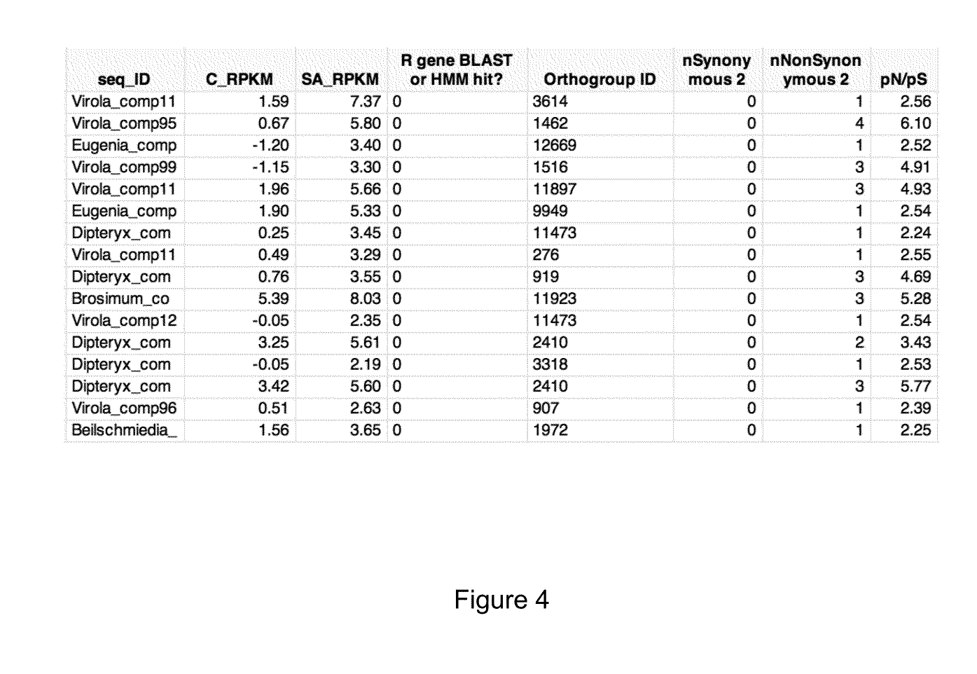

Analyses were done to examine all genes at least four-fold upregulated by salicylic acid treatment compared to control plants, and which have pN/pS>2 (for example). In the six tree species, there were 143 such genes, many of which have known roles in defense responses against pathogens, but also including many genes of unknown function in plant defense. A sample of genes identified by this analysis is shown in FIG. 4.

### Identification of Polymorphisms in Orthologous Gene Across Species

Shared polymorphisms between two or more species may represent potential interesting sites that can be widely generalizable across species. Such shared polymorphisms (i.e. trans-specific polymorphism) can arise from the existence of ancient polymorphisms that predate the divergence of the species In other words, the common ancestor of the two or more species had a polymorphism in a particular gene and both lineages independently maintained that polymorphism after the divergence of the species. Another possibility is that species convergently evolve the same polymorphism at an orthologous site. In either case, it is expected that such identified shared polymorphisms may be important, for example for the pathogen resistance of the species. Thus, they would be of immediate interest to plant breeders or gene editors seeking to improve disease resistance in a plant strain. While there are existing bioinformatic ways to scan genomes of multiple species for such signals of selection (Feulner et al., 2015, PLoS Genetics, 11(2): e1004966), but these require high quality full genome sequence data, and the results are sensitive to demographic history, which tends to be especially problematic for agricultural plants that have gone through severe bottlenecks during selection and propagation of particular favored cultivars.

In contrast to whole-genome scans, the present method allows for the discovery of such trans-specific polymorphisms in more limited (e.g. transcriptome) data. The present method utilizes a targeted approach, using the R gene annotation and polymorphism in the data sets.

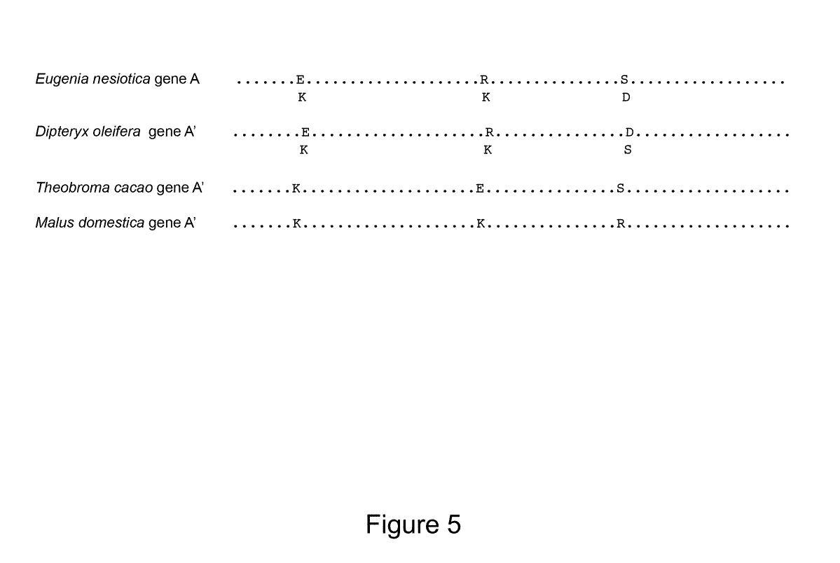

For example, in one analysis, R genes were ranked according to their abundance of non-synonymous polymorphism and it was observed that, in the top-ranked orthogroup of R genes, two long-diverged (probably 10-40 million years or more) species contain an orthologous gene with a number of sites where the two species have the same segregating amino acids (FIG. 5). As shown in FIG. 5, orthologous genes of Eugenia nesiotica and Dipteryx oleifera contain numerous sites where the species have the same segregating amino acids. Further analysis of this gene in several crop species, Theobroma cacao and malus domestica, showed that, in several instances, they have one of the two polymorphic amino acids at the same site in the protein.

### Transcriptome Assembly Statistics

Roughly one third of the assembled gene models contain well supported polymorphism (Table 2; median=7 to 18 polymorphic synonymous sites per thousand synonymous sites) at levels comparable to other wild trees (e.g. 11 SNPs per 1 Kb of coding sequence in pine (Pavy et al., 2013, Genome Biology and Evolution, 5: 1910-1925). Transcriptome-wide synonymous polymorphism was about twice as abundant as non-synonymous polymorphism in all species, indicative of a general history of purifying selection. As observed previously in other species, non-synonymous variation (pN) was about 5 to 10-fold more abundant in R genes than the rest of the transcriptome (Table 2) and concordant with R gene diversity between two genetically distinct strains of rice, where a well-characterized genome guided the assembly and analysis (Yang et al., 2006, Plant Molecular Biology, 62: 181-193).

TABLE 1
Study species and assembly statistics
for non-redundant gene models.

| | N gene | Median | Median read |
|---|---|---|---|
| Species (family) | models | length | depth |
| Virola surinamensis | 26,216 | 753 | 33 |
| (Myristicaceae) | | | |
| Beilschmiedia pendula | 25,747 | 744 | 52 |
| (Lauraceae) | | | |
| Eugenia nesiotica | 24,075 | 957 | 49 |
| (Myrtaceae) | | | |
| Brosimum alicastrum | 19,689 | 1203 | 54 |
| (Moraceae) | | | |
| Lacmellea panamensis | 24,415 | 1491 | 53 |
| (Apocynaceae) | | | |
| Dipteryx oleifera | 30,251 | 627 | 61 |
| (Fabaceae) | | | |

TABLE 2
Polymorphism in non-redundant gene models from root transcriptomes of tropical trees. Data outside
parentheses refer to the transcriptome exclusive of R genes; data in parentheses refer to R genes.

| Species | N monomorphic | N polymorphic | median pN | median pS | median pN/pS |
|---|---|---|---|---|---|
| Virola (Vr) | 18,930 (152) | 7,016 (118) | 0.003 (0.036) | 0.007 (0.058) | 0.54 (0.73) |
| Beilschmiedia (Bl) | 16,556 (241) | 8,795 (155) | 0.004 (0.041) | 0.009 (0.042) | 0.52 (0.84) |
| Eugenia (Eu) | 15,502 (435) | 7,780 (258) | 0.004 (0.022) | 0.009 (0.033) | 0.50 (0.69) |
| Brosimum (Br) | 12,668 (234) | 6,624 (163) | 0.004 (0.019) | 0.009 (0.026) | 0.49 (0.73) |
| Lacmellea (La) | 17,454 (115) | 6,767 (79) | 0.003 (0.024) | 0.006 (0.048) | 0.58 (0.68) |
| Dipteryx (Di) | 18,199 (171) | 11,785 (96) | 0.007 (0.026) | 0.018 (0.043) | 0.46 (0.69) |

### Completeness of Transcriptome Assemblies

To evaluate the completeness of the transcriptome sequence datasets, the frequency of capture of three known conserved sets of plant genes was examined, namely the universally conserved orthologs (UCOs) (Williams et al., 2014, The Plant Cell Online, 26: 2873-2888; Der et al., 2011, BMC Genomics, 12: 99) ultra-conserved Core Eukaryotic Gene (CEG) orthologs from KOGs database (Parra et al., 2007, Bioinformatics, 23: 1061-1067; Parra et al., 2009, Nucleic Acids Research, 37: 289-297), conserved single copy genes from COSII (Solegenomics) and the set of conserved single copy genes sets in 22 representative sequenced plant genomes orthogroups (P. Amborella Genome, 2013, Science, 342: 1241089). The UCO list was obtained from the Compositae genome project (UC Davis) and CEG orthologs from CEGMA projects (UC Davis). The 22 representative plant genomes single copy gene sets were identified by requiring that at least 19 taxa be present in an orthogroup and at least 19 taxa be single copy. The tropical trees transcriptome assemblies were classified into the 22 genomes orthogroups using hmmscan (Eddy et al., 2011, PLoS Computational Biology, 7: e1002195). A conserved single copy orthogroup was considered detected in a tropical trees species if its transcriptome assembly was classified in the orthogroup. Results from this analysis shown in Table 3, Table 4, and Table 5 below indicate that gene coverage ranged from at least 70% in Brosimum to 100% (UCO analysis) in Lacmellea assemblies. Full length recovery of the coding region varied between 76 and 97% for the UCO set (Table 4). These results indicate that the assemblies have excellent gene coverage and are very likely to capture the large majority of genes.

TABLE 3
Transcriptome completeness analysis using 357 Arabidopsis
Eukaryotic ultra-conserved single copy orthologs (UCOs,
The Compositae Genome Project, UC Davis)

| Species | UCOS | Present | % Present |
|---|---|---|---|
| Beilschmiedia pendula | 357 | 347 | 97.2 |
| Brosimum alicastrum | 357 | 274 | 76.8 |
| Dipteryx oleifera | 357 | 353 | 98.9 |
| Eugenia nesiotica | 357 | 353 | 98.9 |
| Lacmellea panamensis | 357 | 357 | 100.0 |
| Virola surinamensis | 357 | 354 | 99.2 |

TABLE 4
Transcriptome completeness analysis using 248
ultra-conserved Core Eukaryotic Gene (CEG) orthologs
(Parra et al., 2007, Bioinformatics, 23: 1061-
1067) from KOGs database (UC Davis)

| Species | CEGs | Complete | % Complete | Partial | % Partial |
|---|---|---|---|---|---|
| Beilschmiedia | 248 | 228 | 91.9 | 238 | 95.9 |
| pendula | | | | | |
| Brosimum | 248 | 188 | 75.8 | 197 | 79.4 |
| alicastrum | | | | | |
| Dipteryx | 248 | 233 | 94.0 | 241 | 97.2 |
| oleifera | | | | | |
| Eugenia | 248 | 229 | 92.3 | 245 | 98.8 |
| nesiotica | | | | | |
| Lacmellea | 248 | 236 | 95.2 | 246 | 99.2 |
| panamensis | | | | | |
| Virola | 248 | 239 | 96.7 | 247 | 99.6 |
| surinamensis | | | | | |

*Partial = Complete genes + Partial genes

TABLE 5
Transcriptome completeness analysis using 22 representative plant genomes
single copy orthogroup sets (P. Amborella genome, 2013, Science, 342: 1241089)

| Single Copy Sets | | | | | % Present | | | |
|---|---|---|---|---|---|---|---|---|
| Taxa | Single Copy | Orthogroups | Beilschmiedia | Brosimum | Dipteryx | Eugenia | Lacmellea | Virola |
| 19 | 19 | 899 | 94.1 | 71.5 | 94.7 | 96.3 | 98.7 | 97.2 |
| 20 | 19 | 864 | 94.4 | 71.4 | 94.9 | 96.5 | 98.7 | 97.2 |
| 20 | 20 | 396 | 95.5 | 72.2 | 95.2 | 96.2 | 99.0 | 98.5 |
| 21 | 19 | 735 | 94.3 | 70.8 | 95.1 | 97.4 | 98.8 | 97.6 |
| 21 | 20 | 364 | 95.1 | 72.5 | 95.3 | 96.4 | 98.9 | 98.4 |
| 22 | 19 | 378 | 94.4 | 71.4 | 94.7 | 97.9 | 98.7 | 98.4 |
| 22 | 20 | 216 | 94.4 | 72.7 | 94.4 | 96.8 | 98.6 | 98.2 |

### Validation of R Gene Assembly:

Verification of Correct Size of Gene Models

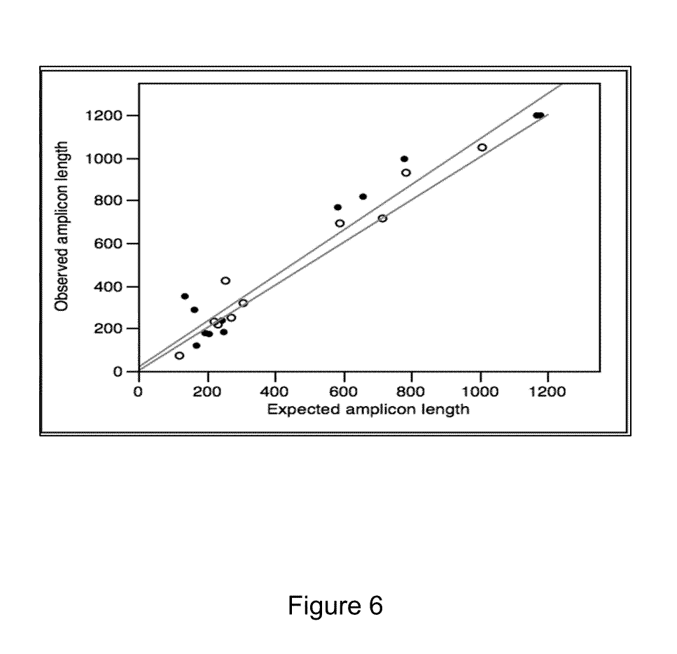

To test the accuracy of R gene assembly, 24 of the most polymorphic R genes less than 1500 nucleotides in length were selected from the Virola (N=13) and Beilschmiedia (N=11) transcriptomes. PCR primers were designed near the ends of these sequences (predicted amplicon length<1200). Observed amplicon sizes (estimated from gels) were compared to predicted sizes. All but one of these genes amplified successfully and all were close to their predicted size (R=0.97) (FIG. 6).

External Consistency of R Gene Polymorphism

If the de novo transcriptome assemblies have accurately estimated levels of R gene polymorphism, the distribution of polymorphism should be similar to what emerges from an equivalent analysis performed using a complete genome sequence. Such an example is available for rice (Yang et al., 2006, Plant Molecular Biology, 62: 181-193).

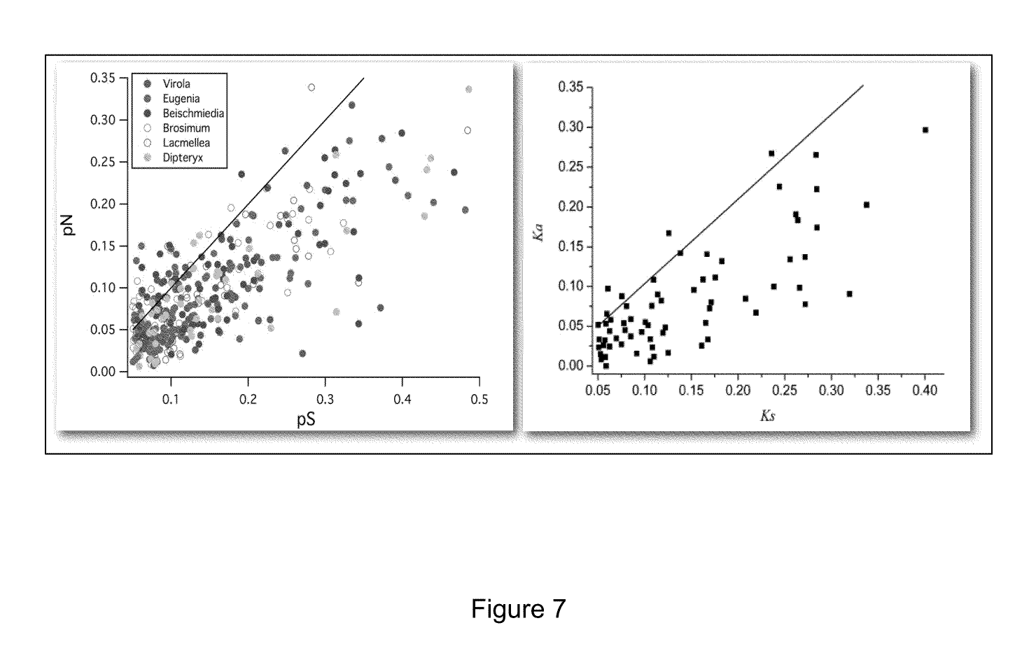

FIG. 7 depicts data comparing the metrics of R gene polymorphism from de novo transcriptome assemblies of tropical trees versus R gene polymorphism obtained from full genome sequences of two rice strains. As shown in FIG. 7, across all six tree species, the de novo assembly described herein produced results indistinguishable from equivalent metrics obtained using a reference genome for assembly and annotation (Yang et al., 2006, Plant Molecular Biology, 62: 181-193).

Note that in the analysis of minor allele frequencies, there is a low abundance of polymorphic sites with allele frequencies close to 50%, as would occur for chimeric assemblies of recently duplicated genes in which presence/absence of the duplication did not also vary among individuals. This is strong evidence that the present analysis did not commonly misassemble recent duplicates.

Cohort Level Replication of pN/pS

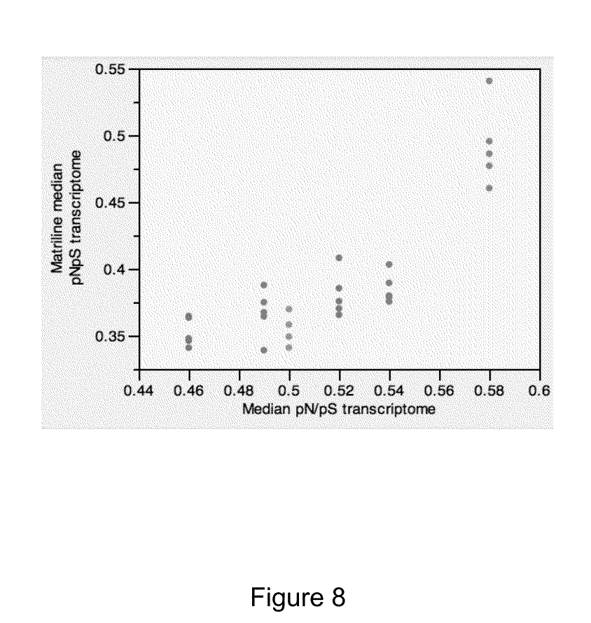

Within each species there was a sufficiently large number of genes (>1K) with sufficient read depth to estimate polymorphism and gene expression metrics when the data were divided into matrilines (N=5 matrilines per species, each comprising 15 seedlings). Here it is shown how pN/pS estimates from matrilines (vertical axis; P<0.0001 for species differences) corresponded with population estimates (horizontal axis; P<0.0001 for correlation) (FIG. 8). It was not possible to do this analysis separately for R genes because too few had sufficient read depth to allow robust estimates of polymorphism and gene expression within individual matrilines. For this reason, species level estimates were used for all analyses, but note that the replicability of transcriptome wide pN/pS indicates that these values reflect the central tendency of relatively invariant 5-fold replicated measures.

Robustness of Median pN/pS

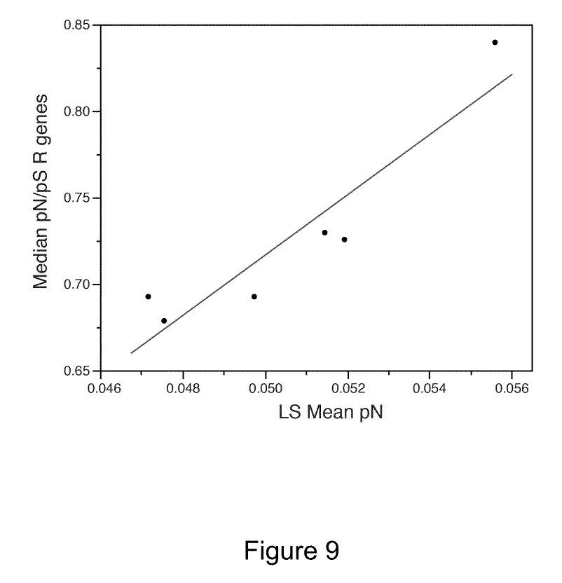

Species estimates of median pN/pS were nearly indistinguishable from LS means for pN obtained from an analysis in which assembly variables (length and average read depth) and the neutral evolution rate (pS) were accounted for (FIG. 9).

The Species term is not significant, but the continuous variable Local Population Size (number of mature size trees) is significantly related to the species mean square pN values that emerge from this model (one-tailed P=0.016).

TABLE 6
Analysis that adjusts R gene pN for pS (neutral
mutation rate) and assembly variables. R² = 0.77.

| | | | Sum of | | |
|---|---|---|---|---|---|
| Source | Nparm | DF | Squares | F Ratio | Prob > F |
| log contig length | 1 | 1 | 0.008 | 9.02 | 0.0027 |
| log ave read depth | 1 | 1 | 0.014 | 16.10 | <.0001 |
| Species | 5 | 5 | 0.008 | 1.86 | 0.0985 |
| pS | 1 | 1 | 1.534 | 1817.81 | <.0001 |

Allele Frequencies

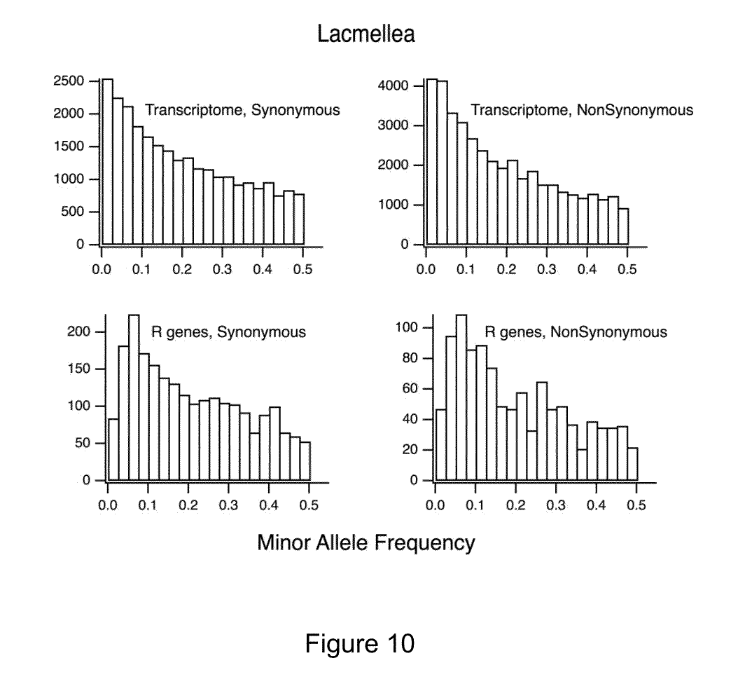

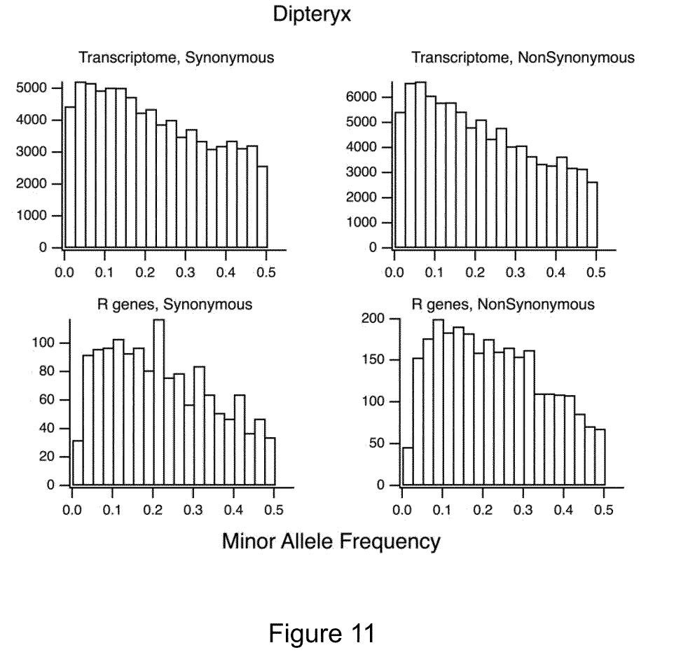

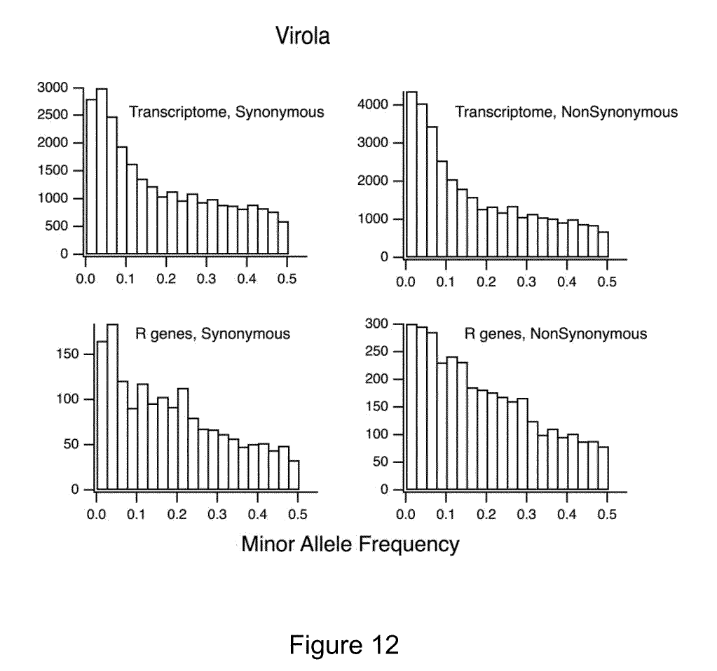

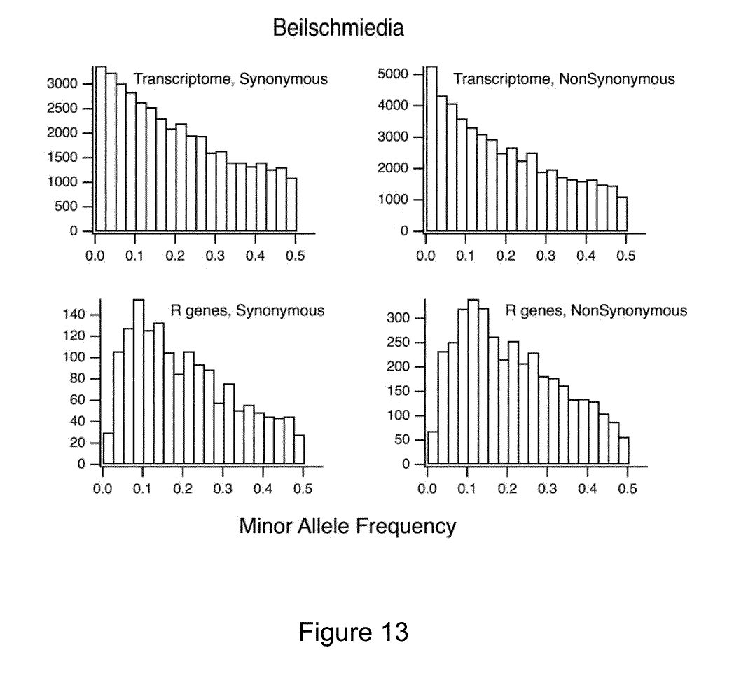

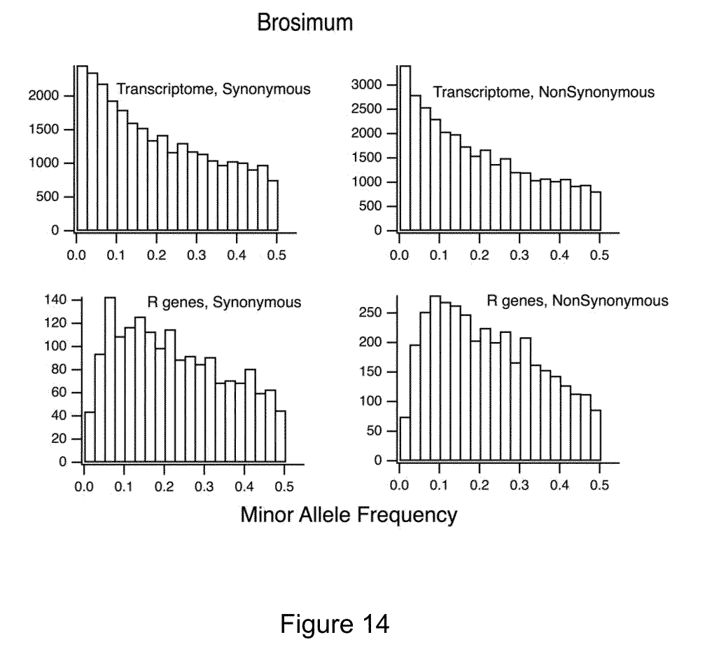

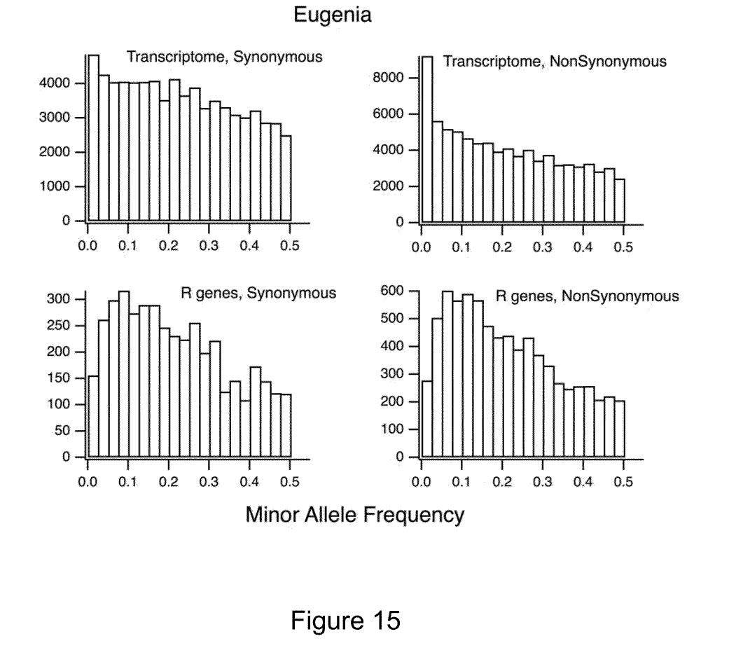

FIG. 10 through FIG. 15 depict the minor allele frequency of synonymous and nonsynonymous variants in the entire transcriptome and in R genes. Note that these data do not show an overabundance of allele frequencies around 0.5, as would occur from chimeric assembly of two distinct genes that do not also vary in presence/absence across individuals. Hence, there is no evidence of frequent chimeric assembly of recently diverged R gene paralogs.

These plots do not directly show evidence for a paucity of rare alleles in R genes because there is strong ascertainment bias in SNP discovery in any transcriptome data. Rare variants are much less likely to be detected in low-expressed genes, whereas both detection of rare variants and sequencing error become much more likely in the most highly expressed genes. R genes tend to be more intermediate in expression and read depth than other genes and this difference influences these site-frequency spectra, including the reduced incidence of rare variants in R genes. For this reason, it is difficult to disentangle the effect of balancing selection from ascertainment bias in these data.

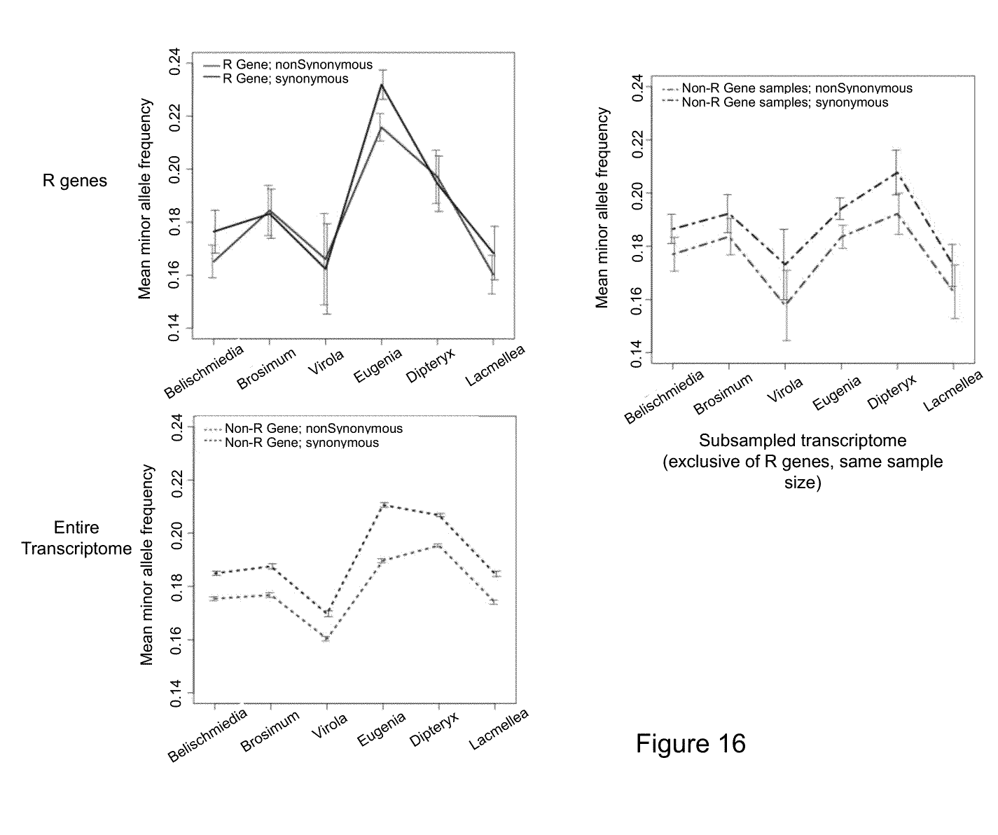

In order to minimize ascertainment biases caused by variation in read depth, an analysis of minor allele frequencies was conducted after excluding the highest and lowest read depth sites, and with equal sampling of R genes and other genes in order to obtain comparable standard errors (FIG. 16). These data show that in the transcriptome exclusive of R genes, there is in all six species a higher minor allele frequency of synonymous variants, as expected when purifying selection predominates. In contrast, R genes show no difference in minor allele frequency of synonymous and non-synonymous sites, as expected under balancing selection, which maintains non-synonymous alleles and linked synonymous variants.

### Detection of Pathogenesis-Related (PR) Genes

In addition to R genes, the gene models were analyzed for the presence and/or expression level of PR genes. Table 6 shows the functional annotations of PR gene families while Table 7 provides the number of PR gene homologs identified in each of the tree species.

TABLE 6
Functional annotations of PR gene families (Campos
et al., 2007, Genet Mol Biol, 30: 917-U198)

| | |
|---|---|
| PR-1 | PR-1a |
| PR-2 | B-1,3-glucanase |
| PR-3 | Chitinase class I, II, IV, V, VI, VII and endochitinase |
| PR-4 | Chitinase Hevein-like |
| PR-5 | Thaumatin-like |
| PR-6 | Proteinase inhibitor |
| PR-7 | Aspartic proteinase |
| PR-8 | Chitinase class III |
| PR-9 | Lignin-forming peroxidase |
| PR-10 | Ribonuclease-like |
| PR-11 | Chitinase, class V |
| PR-12 | Defensin |
| PR-13 | Thionin |
| PR-14 | Lipid transfer protein |
| PR-15 | Oxalate oxidase |
| PR-16 | Oxalate oxidase-like or gemin-like |
| PR-17 | NtPRp27 |

TABLE 7
Number of PR gene homologs identified in each tree species

| Species | N PR genes |
|---|---|
| Beilschmiedia pendula | 202 |
| Brosimum alicastrum | 160 |
| Dipteryx oleifera | 277 |
| Eugenia nesiotica | 172 |
| Lacmellea panamensis | 205 |
| Virola surinamensis | 184 |

### Generation of Candidate Genes

Using these processes, a short list of genes that harbor genetic variation likely to be important in pathogen resistance is generated. For the species in the examples shown above, there was no previous genetic data and nothing known about their genetic-level mechanisms of disease resistance. These analyses were performed for an ecological study, but similar analyses may be done to identify candidate genes to improve durable pathogen resistance in crops. For example, using the data such as those generated by way of the described method, a number of courses of action become available. For example, experimental methods, such as a transient transfection assay can be utilized to confirm the function of the candidate genes in pathogen resistance. A breeding strategy can be enacted to maintain or increase diversity at the identified particular loci in the focal species or cultivar. A breeding strategy can be enacted to maintain or increase diversity at homologous loci in a different species or cultivar. Alleles of the identified genes can be cloned into another species or cultivar. A targeted search for alleles of the homologs of the identified genes can be performed in another species or cultivar. Cloned homologs of the identified genes can be edited in a different species or cultivar in order to introduce novel variation at the loci

Note that the method of uncovering all of the polymorphism in protein coding genes can reveal more than just alleles effective against the “pathogen of the moment”. The ideal solution in crop improvement is to create durable resistance, which likely requires polymorphism at many loci across the genome so that a single mutation in a pathogen strain does not defeat resistance. Hence, a critical part of the process described herein is that it informs the aim of creating durable resistance, a process not readily attainable by other methods.

With these methods, growers can make progress for improving disease resistance in slow-growing plants that are not well suited for methods requiring breeding for multiple generations and/or in plants lacking sequenced genomes. Maximizing diversity (heterozygosity) at these loci is likely to improve plant health in the immediate future (a single generation) and perhaps over the long term as well (durable resistance) by overcoming the loss of diversity in pathogen resistance genes created by bottlenecks, artificial selection, and inbreeding. Alternatively, the information provided by the present method can be used for transgenic approaches, following confirmation of disease resistance efficacy in transient transfection assays.

The process described herein is an improvement over prior methods, as it does not require multiple generations of breeding to map resistance phenotypes, does not require a sequenced genome, and has no a priori limitations about what genes can be examined for variation. These characteristics distinguish this process from methods that require a sequenced genome and/or sets of PCR primers that target sets of already-known genes or gene families (Turner et al., 2010. Nature Genetics, 42, 260-263; Wan et al., 2013. BMC Genomics 2013, 14:109 doi:10.1186/1471-2164-14-109; Vossen et al., 2013. Plant Methods. 2013; 9: 37; Jupe et al., 2013. The Plant Journal, 76: 530-544). PCR-based approaches require a high degree of sequence conservation at the primer-binding sites, which may not exist in disease resistance genes due to their high level of genetic variation and rapid sequence evolution. There is no such requirement with transcriptome sequence. Further, the present method allows for obtaining the full length transcript, whereas PCR-based methods only obtain the region between the PCR primers. Full length coding sequences are needed for functional characterization or functional replacement experiments. More work would be needed to obtain full length coding sequences in the PCR approach. Hence, the present process is a truly unbiased approach that can identify important variation anywhere in the transcriptome (potentially full length recovery of all expressed genes). This method is rapid (ca. 2-4 months from initial sample collection to finished data) and inexpensive compared to other methods for achieving a gene-level and population-level understanding of plant-pathogen interaction across all expressed genes. This process may be the best approach available for plants with long generation times that preclude traditional breeding-based analyses (e.g. QTL and bulk segregant analyses) Because this method does not rely on pre-existing genomic data, this process can identify genes of likely importance for pathogen resistance in wild plant populations, thereby providing information about R-gene diversity in populations never subjected to agriculture (i.e., insights about durable resistance) and hugely increasing the pool of genes available for transgenic methods. These genes could be used directly or serve as models to identify homologous monomorphic loci in bottlenecked and inbred crop plants that could then be made polymorphic by genetic manipulation.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of identifying one or more genes of an organism associated with pathogen resistance or pathogen response, comprising:
identifying the number of polymorphisms in each sequenced contig of a set of sequenced contigs, wherein the set of sequenced contigs are obtained from a pooled RNA sample from one or more sample organisms;
identifying each polymorphism located in the coding region of each sequenced contig as synonymous or non-synonymous polymorphism; and
ranking the sequenced contigs based upon the presence of non-synonymous polymorphism in each sequenced contig.

2. The method of claim 1, wherein the pooled RNA sample comprises the mRNA transcriptome from one or more sample organisms.

3. The method of claim 1, wherein the organism is a plant and where the polymorphisms are identified in each sequenced contig of a set of sequenced contigs obtained from a pooled RNA sample from one or more sample plants.

4. The method of claim 1, further comprising determining if each sequenced contig is a resistance (R) gene.

5. The method of claim 1, further comprising determining if each sequenced contig is a pathogenesis-related (PR) gene.

6. The method of claim 1, further comprising filtering the set of sequenced contigs to provide a set of orthology-established sequenced contigs.

7. The method of claim 1, further comprising determining for each sequenced contig the most homologous gene of a model organism.

8. The method of claim 1, wherein the identified polymorphisms are at least one of single nucleotide variations (SNVs), multiple nucleotide variations (MNVs), and insertion-deletion polymorphisms.

9. The method of claim 1, wherein the sequenced contigs are ranked by the ratio: (number of non-synonymous polymorphisms per non-synonymous site/number of synonymous polymorphisms per synonymous site) (pN/pS).

10. The method of claim 1, further comprising constructing a data structure comprising data for each of the sequenced contigs.

11. The method of claim 10, wherein the data for each of the sequenced contigs comprise at least one of nucleotide sequence, contig name, contig length, read depth, normalized read depth, homologous gene of model organism, description of homologous gene, R gene status, PR gene status, predicted peptide sequence, location of polymorphism, type of polymorphism, number of polymorphisms, number of synonymous polymorphisms, number of non-synonymous polymorphisms, ratio of the number of non-synonymous polymorphisms to the number of synonymous polymorphisms, ratio of number of non-synonymous polymorphisms per non-synonymous site/number of synonymous polymorphisms per synonymous site, ratio of expression under an experimental condition to expression under control condition, ratio of expression under first experimental condition to expression under a second experimental condition, and number of non-synonymous mutations adjusted for contig length and read depth.

12. The method of claim 1, wherein the method comprises comparing the expression level of each sequenced contig in a first sample to the expression level of each sequenced contig in a second sample.

13. The method of claim 12, wherein the first sample is of one or more sample organisms subjected to a treatment and the second sample is of one or more sample organisms not subjected to a treatment.

14. The method of claim 1, further comprising aligning sequenced contigs from two or more species to locate trans-specific amino acid polymorphisms associated with pathogen resistance.

15. The method of claim 1, wherein the identified one or more genes are used to modify an organism to provide the organism with enhanced pathogen defense.

16. The method of claim 15, wherein the organism is modified by at least one of gene editing, introduction of a transgene, or direction of a breeding program.

17. The method of claim 15, wherein modification of the organism also modifies the descendants of the organism.

18. The method of claim 1, wherein at least one of the identified one or more genes is used in the development of an anti-biological compound.

19. A system for identifying one or more genes of an organism associated with pathogen resistance or pathogen response, the system comprising a computing device running a software platform, where the software platform is configured to:
identify the number of polymorphisms in each sequenced contig of a set of sequenced contigs, wherein the set of sequenced contigs are obtained from a pooled RNA sample from one or more sample organisms;
identify each polymorphism located in the coding region of each sequenced contig as synonymous or non-synonymous polymorphism; and
rank the sequenced contigs based upon the presence of non-synonymous polymorphism in each sequenced contig.

20. The system of claim 19, wherein the software platform is configured to construct a data structure comprising data for each of the sequenced contigs, wherein the data for each of the sequenced contigs comprise at least one of nucleotide sequence, contig name, contig length, read depth, normalized read depth, homologous gene of model organism, description of homologous gene, R gene status, PR gene status, predicted peptide sequence, location of polymorphism, type of polymorphism, number of polymorphisms, number of synonymous polymorphisms, number of non-synonymous polymorphisms, ratio of the number of non-synonymous polymorphisms to the number of synonymous polymorphisms, ratio of number of non-synonymous polymorphisms per non-synonymous site /number of synonymous polymorphisms per synonymous site, ratio of expression under an experimental condition to expression under control condition, ratio of expression under first experimental condition to expression under a second experimental condition, and number of non-synonymous mutations adjusted for contig length and read depth.
